(19) United States
(12) Patent Application Publication
(10) Pub. No.: US 2011/0091485 A1
(43) Pub. Date: Apr. 21, 2011

(54) MODULATION OF CELL JUNCTIONS

(76) Inventors: William G. Carter, Bainbridge Island, WA (US); Clarence Dunn, Seattle, WA (US); Elizabeth Wayner, Shoreline, WA (US); Tatiana Zaitsevskaia-Carter, Bainbridge Island, WA (US)

(21) Appl. No.: 12/672,290

(22) PCT Filed: Aug. 6, 2008
(86) PCT No.: PCT/US08/09432

§ 371 Date: Mar. 24, 2010

Related U.S. Application Data
(60) Provisional application No. 60/954,177, filed on Aug. 6, 2007.

(51) Int. Cl. A61K 39/395; A61K 31/185; A61K 31/716; A61K 38/14; A61K 31/7105; A61P 35/00; A61P 29/00; A61P 17/00; A61P 9/00; C12N 5/07

(52) U.S. Cl. 424/174.1; 514/577; 424/172.1; 514/54; 514/20.9; 514/44 R; 514/17.7; 514/19.3; 514/18.6; 514/19.4; 435/375

(57) ABSTRACT

The present invention provides methods of modulating cell junctions via the Gp140 membrane protein. Gp140 agonists and antagonists are provided, including activating and non-activating antibodies of Gp140, as well as compositions including the same. Methods of treatment including administration of a Gp140 agonist or antagonist are also provided. Finally, implants having a Gp140 modulator coupled thereto are provided.

### RELATED APPLICATIONS

This application claims the benefit under 35 U.S.C. §119(e) of U.S. Provisional Patent Application Ser. No. 60/954,177, filed Aug. 6, 2007, the disclosure of which is incorporated herein by reference in its entirety.

### GOVERNMENT FINDING

This invention was made, in-part, with United States government support under grant numbers ROI AR047963 and U54 CA126540 from the National Institutes of Health. The United States government has certain rights to this invention.

### FIELD OF THE INVENTION

This invention describes the modulation of cell junctions by inhibiting or enhancing the actions of Gp140.

### BACKGROUND OF THE INVENTION

“Gp140” is a 140 kDa transmembrane glycoprotein found in various cells. Also called CDCP1, or “Cub Domain Containing Protein 1,” it was found to be associated with and highly expressed in human tumors of the colon, and significantly expressed in human lung carcinomas (Scherl-Mostageer et al. (2001) Oncogene 20:4402-4408). Further characterization suggested that the protein may be selectively upregulated during malignant progression (Hooper et al. (2003) Oncogene 22:1783-1794). Bullring showed expression of Gp140 in hematopoietic stem cells and leukemia (Stem Cells 2004 22:334).

We had previously identified an 80 kDa membrane-associated glycoprotein (p80) involved in anchorage of human keratinocytes and epithelial cells to laminin 5 during wound healing (Xia et al. (1996) J. Cell Biol. 132(4):727-740). We later purified p80, and identified Gp140 as a trypsin-sensitive precursor to p80 (Brown et al. (2004) J Biol. Chem. 279:14772-14783). Plasmin, a serum protease, also converts Gp140 to p80.

Cell junctions, including tight, adherens, focal, desmosomes, hemidesmosome and gap junctions, are dynamic plasma membrane structures found in most animal cell types that mediate communication adhesion, both between cells and between cells and the extracellular environment. In this capacity, they establish the barrier between inside and outside of the host. Gap junctions form aqueous channels that interconnect the cytoplasms of adjacent cells and allow the cells to directly exchange cytoplasmic components (<1000 Daltons) (see, e.g., Lampe et al. (2000) 384(2):205-215). Gap junctions are formed from two hemichannels, each provided by one of the two adjacent cells. Each hemichannel is an oligomer of six connexin proteins. Various members of the connexin family have been identified, and they are generally named based upon the molecular weight of the deduced sequence in kiloDaltons. Gap junctions have rapid turnover rates, and their activity is influenced by posttranslations modifications of the connexin subunits.

Connexin43 (Cx43) is the most widely-expressed connexin in tissues and cell lines, and has been the focus of many phosphorylation studies. Regulation of connexins has been reported to play a role in heart disease (See U.S. Pat. No. 7,153,822 to Jensen et al.), as well as various skin diseases.

### SUMMARY OF THE INVENTION

We have determined that there is a link between Gp140 and multiple cell junctions. Methods of modulating cell junctions of cells are provided herein, including administering a Gp140 agonist or antagonist in an amount effective to modulate cell junctions in vitro or in vivo. In some embodiments agonists include zymosan, Staphylococcus aureus peptidoglycan, and antibodies that signal through the extracellular domain of Gp140 to assemble cell junctions. In some embodiments Gp140 antagonists include antibodies that bind specifically to the extracellular domain of Gp140 but do not activate Gp140. In some embodiments, modulating agents include suramin, ncRNA and soluble peptides. In some embodiments cell junctions are selected from the group consisting of: gap junctions, tight junctions, adherens junctions, desmosomes and hemidesmosomes.

Further provided are methods of modulating a cell junction in a cell, tissue or organ exposed to stress, the method comprising contacting said cell, tissue or organ with a therapeutically effective amount of a Gp140 agonist or antagonist. In general, but without wishing to be bound by theory, Gp140 is a cellular response mechanism, detecting disruption to tissue or organ integrity (e.g., chemical or mechanical integrity, viral insult, etc.). Stress as used herein includes, but is not limited to, a wound of the skin, a cardiac insult, a neuronal insult (e.g., stroke), an inflammatory reaction or condition (e.g., cystic fibrosis), etc.

Also provided are methods of treating a cell junction disorder, including administering to said subject a Gp140 agonist or antagonist in an amount effective to treat said cell junction disorder. In some embodiments, the subject is in need of treatment for a cell junction disorder selected from the group consisting of: a connexin disorder (e.g., skin disorders, nervous system disorders, heart disorders or muscle disorders), a tight junction disorder, an adherens junction disorder and a desmosome disorder.

Further provided are methods of treating a cancer in a subject in need thereof, including administering to said subject a Gp140 agonist or antagonist in an amount effective to treat said cancer, consistent with (but not intended to be bound by) the theory of cancer as a response of the body to chronic stress and inflammation. In some embodiments, the cancer is selected from the group consisting of: skin cancer, lung cancer, prostate cancer, breast cancer and colon cancer.

Compositions comprising a Gp140 agonist or a Gp140 anatagonist in a pharmaceutically acceptable carrier are also provided.

A further aspect of the present invention is an activating Gp140 antibody that specifically binds to the extracellular domain of Gp140. Also provided are non-activating antibodies and blocking antibodies of Gp140.

An implant, e.g., a stent or ophthalmic implant, having an active agent coupled thereto is also provided.

Another aspect of the present invention is the use of a Gp140 modulator as described herein for the preparation of a composition or medicament for carrying out a method of treatment as described herein, or for making an article of manufacture as described herein. Further provided is the use of a Gp140 modulator (agonist or antagonist) for treating a wound, a cell junction disorder, a cardiac disease, a cancer, a brain trauma or injury, an inflammatory disease, or other cell, tissue or organ stress.

### BRIEF DESCRIPTION OF THE DRAWINGS

FIG. 1. Gp140 localizes in cell-cell contacts of quiescent epidermis and is absent from cell-substrate contacts with the basement membrane. (left) Staining of normal epidermis with anti-Gp140 mAb. Arrows indicate basement membrane. (right) Staining of Gp140 (red) and nuclei (blue with DAPI).

FIG. 2. (A) Cx43 immunofluorescence in LA25 cells that had been transfected with empty vector (EV) or Gp140 and grown at the permissive 35° C. Note the punctate membrane staining reminiscent of gap junctions in the latter case. (B) Dye transfer is significantly reduced (p<0.001) at the src permissive temperature of 35° C. but not when Gp140 is present or at 40° C.

FIG. 3. Zymosan or activating anti-Gp140 mAb (ActGp140 P1C3) change the organization of Gp140 by assembling a Triton-X100-resistant Gp140 membrane cluster. Zymosan or ActGp140 mAb were added to keratinocytes and incubated (30 min), washed, fixed (2% formaldehyde in cacodylate, 10 min) and permeabilized (0.5% Triton-X100, 5 min).

FIG. 4. Both activating anti-Gp140 mAbs (P3D9) and non-activating anti-Gp140 mAbs (P5H10) selectively immunoprecipitate Gp140.

FIG. 5. Contact of zymosan with keratinocytes assembles components of the Par polarity complex and tight junctions into cell-cell contacts that can exclude microbes in host defense. Human keratinocytes were grown in 30 μM Ca⁺² then treated with zymosan (20 μg/10⁶ cells) for 30 min or 2 h, extracted with Triton X-100 (0.5% for 5 min) and fixed and stained with Abs against ZO-1 and afadin (AF-6).

FIG. 6. ActGp140 mAb (P3D9) recruits ZO-1, atypical PKC (aPKC) and Cx43 into a Triton-resistant cell-cell junctions. Keratinocytes were treated (2 h) with ActGp140 mAb, washed, extracted with Triton X-100 detergent (0.5%, 5 min, 4° C.), fixed and stained with Abs against the indicated proteins. After 2 h treatment the ActGp140 mAb has internalized Gp140 into cytoplasmic vesicles (panel B).

FIG. 7. ActGp140 mAb binding to keratinocytes recruits Tiam1, a Rac GEF, to cell-cell contacts.

FIG. 8. Zymosan increases internalization of Gp140 via a SFK-dependent mechanism. Zymosan (20 μg/106 cells) was added to human keratinocytes and incubated (2 h) + or − an inhibitor of SFKs (PP2, 5 μM).

FIG. 9. (A) Human keratinocytes were treated as indicated (HK treatment) with an activating anti-Gp140 Mab (a-Gp140, P1C3, 1 ug/ml, 20 min) or zymosan (1 mg/ml, 20 min) or untreated, extracted with Triton X-100 and immunoblotted with anti-pY Mab (4G10) or anti-Gp140pY734 Ab (α-Gp140pY734). Migration of Y phosphorylated proteins (Gp140, FAK etc) are indicated. Both anti-Gp140 Mab and zymosan dramatically increase Gp140 phosphorylation above the level of the untreated lane. (B) Extracts in (A) from untreated, Zymosan and anti-Gp140 mAb treated cells were immunoprecipitated (IP condition) with Abs: anti-pY Mab (4G10, Total pY), WGA (binds sialylated membrane glycoproteins) and anti-Gp140 Mab (α-Gp140, CU_{B4}) or negative control (Nol° Ab). The immunoprecipitates were then immunoblotted with anti-Gp140pY734 Ab or anti-paxillin pY118 Abs. (C) Quantitation of Gp140 phosphorylation on Y734 in response to increasing dosage of zymosan.

FIG. 10. ActGp140 mAb increases phosphorylation of Gp140 at Y734, PKCδ and SFK at Y416. PC3 prostate cells were either untreated or treated with suramin, ActGp140 mAb (P3D9) or NActGp140 mAb (P5H10), and examined directly by immunoblotting of cell lysates (Lysate) or first immunoprecipitated with anti-Gp140, anti-pY or anti-PKCδ Abs (designation under IP:) prior to blotting. Blots were examined with anti-Y, PKCδor SFKpY416 Abs (designated under Blot).

FIG. 11. ActGp140 mAb, but not NactGp140 mAb, recruits PKCδ to cell-cell contacts. Keratinocytes (grown in 30 μM Ca⁺²) were untreated or treated with ActGp140 or NActGp140 mAbs for 1.5 hrs, washed, extracted with Triton X-100 (0.5% at 4° C. for 5 min) then stained.

FIG. 12. Phosphorylation of Gp140Y734 by Src promotes internalization. Wild type (wt) human Gp140 cDNA was transfected into LA25 cells expressing a temperature sensitive Src. The expressed Gp140 was detected with an anti-Gp140 Mab (P1C3) that reacts with human, but not rat, Gp140. (Left) At 40° C., the non-permissive temp for Src kinase activity, wt Gp140 was expressed on the cell surface. (Middle) At 35° C., the permissive temp for Src kinase activity, wt Gp140 was phosphorylated and internalized into the cytoplasmic. (Right) Gp140Y734F expressing a Y to phenylalanine (F) mutation at Y734F did not internalize at 35° C., the permissive temp. Red, Gp140. Blue, DAPI.

FIG. 13. Src inhibition increases gap junctional density at wound cell-cell interfaces. Keratinocytes were pretreated with PP2 (5 μM, 2 h), scratch wounded and after 12 h were fixed in cold methoano/acetone and processed for immunofluorescence for total Cx43 (green). Cell nuclei were stained with DAPI (blue) and the scratched area is shown by an arrow.

FIG. 14. Keratinocytes become highly phosphorylated on S279/282 of Cx43 within 30 min of contact with ActGp140 mAb. Keratinocytes were exposed to ActGp140 mAb and cells were harvested for immunoblotting at 0-24 h. Parallel blots were probed for total Cx43 and Cx43 phosphorylated at S279/282.

FIG. 15. Time course of the change in Cx43 phosphorylation levels. The ratio of phosphorylated Cx43 to total Cx43 for pS279/282, pY247 and pY247 and pS368 is plotted—time points at 0.5, 1, 3, 6, 24 and 48 h were assayed.

FIG. 16. (A) Src activity leads to reduced levels of Cx43 in gap junctions unless rescued by ALLN treatment. LA25 cells grown at 40° C. or 35° C. (permissive) were untreated or treated with ALLN for 3 h and total Cx43 levels were determined by immunoblot. (B) ALLN treatment increases the levels of Y247 phosphorylation relative to total Cx43 when src is active. Cells grown at 35° C. were treated with ALLN for 3 h or untreated and pY247 and pY265 levels relative to total Cx43 were determined. In both A & B total cell lysates were harvested, blotted and probed (n=4 separate expts).

FIG. 17. pY247 staining occurs in the center of large gap junction plaques in the presence of ALLN. Cells were treated with ALLN for 3 h and immunostained for total (green) and pY247 (red) Cx43. pY247 is present in the center of larger gap junctions and predominates in an apparent internalized gap junction (indicated by arrow). X-y, x-z and y-z dimensions all indicate a higher concentration of pY247 in the center portion of the gap junctions (arrowheads indicate x-z and y-z slice planes).

FIG. 18. Gp140 is phosphorylated in cell-cell contacts but dephosphorylated and internalized at the wound edge. (A-B) Incision wounds in human skin were excised 1 h after injury, frozen, cryostat sectioned, extracted without (A, −TX, bar=25 μM) or with (B, +TX; arrow indicates wound edge) Triton X-100 (1.0%, 10 min) then fixed and stained with anti-Gp140 mAb (P1C3). (C-D same field; bar=20 μM) Cultured keratinocytes were fixed and permeabilized with saponin (0.1%, 5 min) and stained with either anti-Gp140 mAb (P3D9) or anti-SFKpY416 Ab. (E-F; bar=40 μM) Confluent keratinocytes were scrape wounded and treated (G, −PP2) without or (H, +PP2) with an inhibitor of SFKs (PP2, 5 μM, overnight) to prevent internalization of the Gp140 vesicles. (G, upper) Keratinocytes were grown at sparse (Sp; lane 1 and 4) or confluent (Con; lane 2 and 5) cell densities for 3 days in keratinocyte basal medium (KBM) or KBM with epidermal growth factor (KGM) then fractionated by SDS-PAGE and immunoblotted with anti-Gp140pY734 Ab. In controls, confluent keratinocytes were also treated with suramin (Con+Sur; lane 3 and 6; 35 μM, 20 min) to maximize the phosphorylation of Gp140. (G, lower) Keratinocytes grown at confluence (Con) were scrape wounded, fractionated and immunoblotted with for anti-Gp140pY734 Ab.

FIG. 19. Cx43 pY265 immunostaining (red) of a human skin 6 h postwounding. The wound edge is indicated by the large arrow. Cytoplasmic/perinuclear staining is indicated by the arrowhead and gap junctional-appearing staining by small arrow. DAPI is in blue.

FIG. 20. Ligation of Gp140 with ActGp140 mAb-beads assembles a detergent-resistant Gp140 membrane cluster and increases phosphorylation of Gp140 and SFK at the site of bead contact. Beads (4 μM) coated with ActGp140 mAb (P3D9) were adhered to the apical surface of keratinocytes and then extracted with Brij99 detergent (0.1%, 5 min) to remove cell components not bound by ActGp140-beads. Cells were fixed (2.0% formaldehyde for 10 min) and stained as indicated. Co-distribution is yellow.

FIG. 21. ActGp140 mAb or suramin increases phosphorylation and assembly of Gp140 in detergent-resistant Gp140 membrane clusters in cell-cell contacts detected with cross-linking. (A a-d; bars=20 μM) Keratinocytes were grown in low Ca⁺² (30 μM) then treated (a) without or (b-d) with anti-Gp140 mAb (P1C3) for 15 min., washed, fixed, permeabilized with 0.5% Triton and stained with Abs. (b-c) anti-Gp140 mAb (P1C3). (d) Same field as c stained with Abs against Gp140pY734. (B) human keratinocytes were treated with (+) or without (−) suramin (35 μM, 15 min) then sequentially extracted with 1% Tx-100 (Tx) and 1% SDS to solubilize the Triton-insoluble material (TxI) and those extracts were subjected to SDS-PAGE and immunoblotted with anti-Gp140pY734 Ab. (C) Tx-100-soluble extracts from HKs treated with (+) or without (−) suramin for 20 min and with (+) or without (−) 2% formaldehyde for the last 10 min of the suramin incubation period were immunoblotted with anti-Gp140 pY734 Ab. In the TX100-soluble fractions, the cross-linked clusters were identified as discrete bands. (D) Suramin-treated HKs were treated without (−) or with (+) the cleavable, membrane impermeable cross-linker DTSSP prior to extraction, SDS-PAGE and immunoblotting with anti-Gp140 Ab. Clusters observed during non-reducing conditions could be cleaved to yield Gp140 and/or p80 upon reduction with 2-mercaptothanol.

FIG. 22. (A) HKs were nucleoporated in suspension with Gp140 siRNA 232 or with no siRNA, then re-adhered to cover slips and grown for 96 hrs. Indicated samples were treated with suramin (35 μM for 1 hr) to promote phosphorylation. Greater than 90% of the cells completely down-regulated Gp140 regardless of the presence or absence of suramin. Only occasional colonies of cells (shown) continued to express Gp140. (B) Comparison of mRNA transcripts expressed in control HKs and HK treated with Gp140 siRNA to down-regulate Gp140 and Gp140-dependent transcripts.

FIG. 23. Specificity of the pS279/282 antibody. 293T cells expressing wild type (WT) or S279/282A mutant Cx43 (MUT—parental 293T cells only express low levels of endogenous Cx43) treated with PDGF, EGF or TPA. Note that TPA and EGF significantly increase binding of the antibody and that S279 and 282 are required.

FIG. 24. Specificities of the pY247 and pY265 antibodies. LA-25 cells expressing active v-src at the permissive temperature (35° C.) react with the pY247 antibody to Cx43 when no peptide or the pY265 immunizing peptide but not when the pY247 peptide was included in the incubation of the Western blot or when cells were at the non-permissive temperature (40° C.). Similarly, the pY265 antibody reacted only at the permissive temperature in the absence of its immunizing peptide.

FIG. 25. Signals through Gp140/CDCP1 promote α-catenin in cell-cell contacts. Human keratinocytes were grown in low Ca⁺² (30 μM) in order to disassemble adherens junctions that contain cadherins and α-catenin. The keratinocytes were treated without (−Left panel) or with (+Right Panel) Gp140 siRNA to knockdown expression of Gp140. (A-C) Without Gp140 siRNA, Gp140 was expressed and overlapped with α-catenin in cell-cell contacts (arrows) as detected by double staining with antibodies against Gp140 (Green) and α-catenin (Red; A-C, D-F, G-I and J-L are the same fields). (D-F) Activation of Gp140 with suramin (35 3 hrs) first increased localization of Gp140 with α-catenin in cell-cell contacts (not shown) followed by internalization of Gp140 into the cytoplasm and a dramatic accumulation of α-catenin in cell-cell contacts (arrows). (G-L) However, with Gp140 siRNA, Gp140 was downregulated and α-catenin was not localized in cell-cell contacts either without suramin (G-H) or with suramin activation (J-L). Arrows indicate minor subpopulation of cells where Gp140 was not knocked down surrounded by the majority of Gp140 was knocked down. Only in cell-cell contacts where Gp140 was retained did α-catenin localize in cell-cell contacts (J-K arrow).

FIG. 26. Suramin (Sur) and anti-gp140 mAb (P3D9) signal through Gp140 to activate SFK(s) and PKCS that phosphorylate substrates that mediate cell-cell adhesion. First, keratinocytes were treated without (−) or with (+) Gp140 siRNA (RNAi) to knockdown expression of Gp140. Knockdown of Gp140 protein (prot) was confirmed by ELISA assay or immunoblotting with antibodies against Gpl40pY734 as indicated. Second, activation of Gp140 with either suramin (5 min, 35 mM) or activating anti-Gp140 mAb (P3D9, 5 min) increased phosphorylation of Gp140Y734, SFKY416 and PKCdY311 by SFKs. PKC activity was also increased as determined by phosphorylation of VaspS157, a substrate for PKCδ. A protein band that co-migrates with α-catenin was phosphorylated by PKC as determined by immunoblotting with an antibody the binds to protein substrates that are phosphorylated on serine residues by PKC (PKC pS sub). Third, knockdown of Gp140 prevented phosphorylation of substrates for SFKs and PKC in response to either suramin or P3D9.

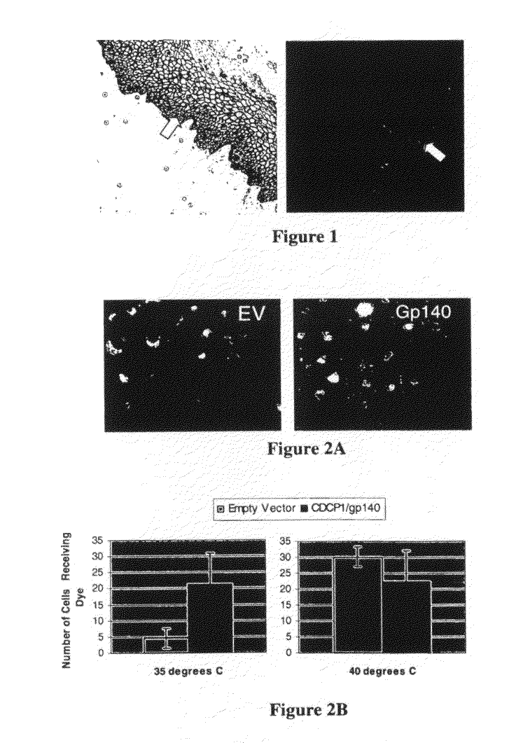

### DETAILED DESCRIPTION OF THE PREFERRED EMBODIMENTS

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Applicants specifically intend that all United States patent references cited herein be incorporated herein by reference to the extent they are consistent with the disclosures herein.

“Gp140” is a 140 kDa transmembrane glycoprotein found in various cells. It was initially identified as a proteolytic fragment of Gp140 termed “p80,” a cell surface protein that is phosphorylated on tyrosine (Y) residues in response to changes in cell adhesion in epidermal wounds (Xia et al. (1996)). Also called CDCP1, or “Cub Domain Containing Protein 1,” it was found to be associated with and highly expressed in human tumors of the colon, and significantly expressed in human lung carcinomas (Scherl-Mostageer et al. (2001) Oncogene 20:4402-4408). Further characterization suggested that the protein may be selectively upregulated during malignant progression (Hooper et al. (2003) Oncogene 22:1783-1794). Buhring showed expression on hematopoietic stem cells and leukemia (Stem Cells 2004 22:334). See also U.S. Patent Publication No. 20070031419 to Domon et al. and U.S. Patent Publication No. 20070009543 to Burgess et al.

“Cell junctions” are proteinaceous structures that connect cells to adjacent cells (i.e., cell-cell junctions) and/or to the extracellular matrix (i.e., cell-ECM junctions). Examples of cell-cell junctions include, but are not limited to, gap junctions, tight junctions, adherens junctions, desmosomes, etc. Examples of cell-ECM junctions include, but are not limited to, focal adhesions, hemidesmosomes and integrin complexes. “Cell junction disorders” include, but are not limited to, connexin disorders, tight junction disorders, adherens junction disorders, focal adhesion disorders, desmosome disorders and hemidesmosome disorders. Cells may be in vitro (e.g., for tissue culture applications) or in vivo (e.g., for methods of treatment). Inherited (genetic) acquired (autoimmune, wounds) defects in cell junctions disrupt functional integrity of the organism and/or predispose the organisms to malignancy.

“Gap junctions” are dynamic plasma membrane structures found in most animal cell types (see, e.g., Lampe et al. (2000) Archives of Biochem. and Biophys. 384(2):205-215), particularly skin, nervous tissue, heart and muscle. They form aqueous channels that interconnect the cytoplasms of adjacent cells and allow the cells to directly exchange water, ions and small molecules (<1000 Daltons). They are involved in quick, short range messaging between cells, termed “gap junction communication” or “GJC.”

Gap junctions are formed from two hemichannels, each provided by one of the two adjacent cells. Each hemichannel is an oligomer of six connexin proteins. The hemichannels form a tight seal, and is normally associated with a 2-3 nm gap between the cell membranes. Various members of the connexin family have been identified, and they are generally named based upon the molecular weight of the deduced sequence in kiloDaltons. Gap junctions have rapid turnover rates, and their activity is influenced by post-translation modifications of the connexin subunits. “Connexin43” or “Cx43” is the most widely-expressed connexin in tissues and cell lines. Its regulation has been implicated in many disorders of the skin and heart.

“Connexin disorders” are ailments involving a disruption in gap junction structure (e.g., membrane localization and/or clustering) and/or function (e.g., GJC) caused by a mutation in one of the connexin genes. Connexin disorders that feature skin abnormalities include, but are not limited to, keratitis-ichthyosis deafness syndrome, erythrokeratoderma variabilis, Vohwinkel's syndrome, and hypotrichosis-deafness syndrome. Connexin disorders of the heart include, but are not limited to, cardiac arrhythmias. Connexins disorders also include non-syndromic deafness, Clouston syndrome, polyneuropathy, cataracts, X-linked Charcot-Marie-Tooth syndrome, erectile dysfunction, etc. See U.S. Pat. No. 7,153,822 to Jensen et al.

We have determined that Gp140 and Cx43 are linked in the following ways:

1) Activation of Gp140 activates src family kinases (SFKs) that increases tyrosine phosphorylation of Gp140 on Y734, protein kinase C delta (PKCδ) on Y311 and src on Y416 while decreasing GJC in keratinocytes at the wound edge.

2) Cx43 present in keratinocytes at the wound edge is phosphorylated on S279/282, Y247, Y265 and S368 in response to wounding or ligation of Gp140 by ActGp140 mAbs.

3) Ligation of Gp140 through a specific monoclonal antibody or the presence of microbes (zymosan) increases gap junction assembly and GJC except when src is active. Inhibition of src promotes gap junction formation and GJC in primary keratinocytes.

4) Transfection of LA-25 cells that contain temperature sensitive src with mutant and wild type Gp140 results in decreased GJC at the permissive temperature in cells with the mutant Gp140 but the wild type Gp140 expressing cells retain GJC even in the presence of active src.

5) Inhibition of src kinases with PP2 or low calcium leads to a dramatic increases in cell surface Gp140 and junctional Cx43 and dye transfer in keratinocytes.

6) Phosphorylation of Cx43 on Y247 appears to be a signal for internalization of Cx43 from the center of large gap junctional plaques.

We have also found that Gp140 signaling is involved in the formation of cell-cell adhesion, such as tight junctions. For example, in some embodiments the activation of Gp140 promotes the formation of tight junctions by, e.g., activating SFK(s) and PKCδ that phosphorylate substrates that mediate cell-cell adhesion.

These data lead us to hypothesize that Gp140 is a novel sentinel in cell-cell contacts of various tissues, including quiescent epidermis that responds to wounds or microbes, resulting in changes in GJC via Cx43-containing junctions as well as cell-cell adhesion, which serves as a barrier function to protect against invading microbes.

“Tight junctions,” also known as “zonula occludens,” are membrane protein complexes involved in cell-cell adhesion, and leave little space (<1 nm) between cell membranes, effectively blocking the passage of molecules and ions through the space between the cells. They selectively modulate permeability between extracellular compartments, and maintain epithelial cell polarity by blocking the movement of integral membrane proteins between the apical and basolateral surfaces of the cell. Materials must actually enter the cells in order to pass through the tissue, providing control over which materials are allowed through. Examples of tissues in which tight junctions play and important role include, but are not limited to, the epidermis of the skin and the blood brain barrier. Proteins involved in the formation of tight junctions include occludin, claudin 1, e-cadherin, ZO-1, JAM-1, catenins, cingulin, and actin. “Tight junction disorders” are disorders involving the structure and/or function of tight junctions. Examples of tight junction disorders include, but are not limited to, deafness, autoimmune disorders, primary hypomagnesemia, and gastrointestinal inflammation. See U.S. Pat. No. 6,458,925 to Fasano. The down-regulation of tight junctions has also been implicated down-regulated in patients with chronic venous insufficiency.

“Adherens junctions,” or “zonula adherens,” are membrane complexes which include cadherins, β-catenin and α-catenin. They can be found in epithelial cell-cell junctions, and are normally more basal than tight junctions. “Adherens junction disorders” are disorders involving the structure and/or function of adherens junctions.

“Focal adhesions,” or “cell-matrix adhesions” are dynamic macromolecular protein assemblies through which the cell cytoskeleton connects to the extracellular matrix (ECM). They mediate cell anchorage of extracellular matrix adhesion on cell behavior, and provide about 15 nm between the plasma membrane and the ECM substrate. They can contain over 100 different proteins, and their attachment to the ECM generally involves the integrins. “Focal adhesion disorders” are disorders involving the structure and/or function of focal adhesions. “Desmosomes,” or “macula adherins,” are membrane complexes which include cadherins and keratins of the cytoskeleton, attaching cell surface proteins to the cytoskeleton. They help epithelial cells resist shearing forces. They leave a space between cells of about 30 nm. “Hemidesmosomes” are similar in appearance to desmosomes when viewed by electron microscopy, but rather than linking two cells, they attach a cell to the extracellular matrix using integrin cell adhesion proteins. “Desmosome disorders” are those involving the structure and/or function of desmosomes and/or hemidesmosomes. Examples of desmosome disorders include, but are not limited to, blistering diseases such as Pemphigus vulgaris.

“Active agent” or “modulator” as used herein refers to a compound that affects the structure and/or function of cell junctions. According to the present invention, this may be accomplished by, e.g., affecting the structure and/or function of Gp140 (also known as CDCP1). Examples of modulators according to the present invention include, but are not limited to, a direct agonist or antagonist of Gp140 (i.e., binding to one or more sites and directly affecting Gp140, by, e.g., itself activating or antagonizing Gp140, or blocking the effects of another agent, etc.) and an indirect agonist or antagonist of Gp140. For example, in some embodiments Activating Gp140 monoclonal antibodies activate src family kinases (SFKs) and increase SFK-dependent phosphorylation of SFK, Gp140, PKCδ and Cx43. They also promote assembly of cell-cell junctions. Examples of other Gp140 agonists include microbial compounds such as zymosan and Staphylococcus aureus peptidoglycan, and in some embodiments the binding of zymosan and/or Staphylococcus aureus peptidoglycan also results in these phosphorylation and functional changes (e.g., in keratinocytes). Other examples of Gp140 modulators include, but are not limited to, suramin, PKC activators such as TPA (tetradecanyl phorbol acetate) (see U.S. Pat. No. 5,962,504 to Kozikowski et al.), SRK activators (see Abdel-Ghany et al., “Control of src kinase activity by activators, inhibitors, and substrate chaperones,” Proc. Natl. Acad. Sci. 87:7061-7065, 1990), h_{y}drogen peroxide, non-coding RNA (e.g., an siRNA, shRNA, miRNa, etc., targeting, e.g., Gp140, either directly or indirectly), soluble peptides (e.g., recombinant extracellular CUB domains, recombinant extracellular domain of Gp140, etc.) and small molecule agonists and antagonists. In some embodiments active agents (e.g., recombinant CUB domains, Gp140 antibodies, etc.) are provided on beads (e.g., fluorescent beads), which prevents internalization by the cells.

“Microbial compounds” include, but are not limited to, Gram-negative and Gram-positive bacterial cell wall compounds such as lipopolysaccharide and peptidoglycan (e.g., Staphylococcus aureus peptidoglycan), and the yeast cell wall polysaccharide zymosan.

“Suramin” is a polysulfonated naphthylurea compound that has been widely used for the treatment of trypanosomiasis (sleeping sickness) and onchocerciasis since the early 1920s. It has recently been shown to block the activity of several types of growth factors, and in some contexts has antineoplastic activity. Suramin treatment of mouse epidermis induces phosphorylation and proteolytic cleavage of Gp140 to p80 (Brown et al., “Adhesion or plasmin regulates tyrosine phosphorylation of a novel membrane glycoprotein p80/gp140/CUB domain-containing protein 1 in epithelia,” J. Biol. Chem. 279: 14772-83, 2004).

“Non-coding RNA” or “ncRNA” as used herein includes both natural and synthetic ncRNAs. Examples include, but are not limited to, small interfering RNA (siRNA), micro RNA (miRNA), piRNAs, ribosomal RNA (rRNA), small nuclear RNA (snRNA), small non-mRNA (snmRNA), small nucleolar RNA (snoRNA), small temporal RNA (stRNA) and other RNAs that regulate the function of mRNAs. See, e.g., D. Bartel et al., PCT Application Publication No. WO 2005/102298; see also T. Kowalik et al., US Patent Application Publication No. 20050186589. Some ncRNAs may be in the form of a natural or synthetic short hairpin RNA or “shRNA,” which short hairpin RNA may or may not be subsequently processed to form a mature ncRNA. In general, ncRNAs as used herein may be any suitable length, but are typically short, e.g., from 5, 10 or 15 nucleotides in length, up to 25, 30 or 35 nucleotides in length. Nucleic acids encoding ncRNAs as used herein may be natural or synthetic and may be derived from any suitable source, including plant, animal, and microbe sources as described herein.

“Small interfering RNA” or “siRNA” (sometimes also referred to as short interfering RNA or silencing RNA) as used herein has its ordinary meaning in the art. In general, siRNAs are double-stranded RNA molecules that are 15 or 20 nucleotides in length, up to 25 or 30 nucleotides in length. siRNAs are known. See, e.g., U.S. Pat. Nos. 7,101,995; 6,977,152; and 6,974,680.

“MicroRNA” or “miRNA” as used herein has its ordinary meaning in the art. Typically, a miRNA is an RNA molecule derived from genomic loci processed from transcripts that can form local RNA precursor miRNA structures. The mature miRNA usually has 20, 21, 22, 23, or 24 nucleotides, although in some cases it may include a greater of lesser number of nucleotides, for example, between 18 and 26 nucleotides. The miRNA has the potential to pair with flanking genomic sequences, placing the mature miRNA within an imperfect RNA duplex which may be needed for its processing from a longer precursor transcript. In animals, this processing may occur through the action of Drosha and Dicer endonucleases, which excise a miRNA duplex from the hairpin portion of the longer primary transcript. The miRNA duplex comprises the miRNA and a similar-sized segment known as the miRNA* (miRNA star) from the other arm of the stem-loop. See, e.g., US Patent Application Publication No. 20060185027.

“Soluble peptides” include, but are not limited to, recombinant extracellular CUB domains, recombinant extracellular domain of Gp140, etc. See, e.g., U.S. Patent Publication No. 20070031419 to Domon et al. and U.S. Patent Publication No. 20070009543 to Burgess et al.

“Antibody” or “antibodies” as used herein refers to all types of immunoglobulins, including IgG, IgM, IgA, IgD, and IgE. The term “immunoglobulin” includes the subtypes of these immunoglobulins, such as IgG₁, IgG₂, IgG₃, IgG₄, etc. Of these immunoglobulins, IgM and IgG are preferred, and IgG is particularly preferred. The antibodies may be of any species of origin, including (for example) mouse, rat, rabbit, horse, or human, or may be chimeric or humanized antibodies. The term “antibody” as used herein includes antibody fragments which retain the capability of binding to a target antigen, for example, Fab, F(ab′)₂, and Fv fragments, and the corresponding fragments obtained from antibodies other than IgG. Such fragments are also produced by known techniques.

In some embodiments, the antibodies are “Gp140” antibodies, in that they specifically bind to Gp140, as measured by the ability of the antibodies to immunoprecipitate the Gp140 protein.

In some embodiments, antibodies are “activating” Gp140 antibodies (“ActGp140”), in that they activate the actions of Gp140. In some embodiments, the binding of activating antibodies 1) promotes the assembly of Gp140 membrane clusters; 2) activates SFKs and increases phosphorylation of Gp140, SFK and PKCδ by SFK; 3) recruits PKCδ to the detergent-resistant Gp140 membrane cluster as a complex with Gp140; 4) promotes assembly of GJs and/or tight junction proteins; 5) increases phosphorylation of Cx43 at S279/282 by MAPK, and/or at S368 by PKC and/or Y247/265 by SFK; or 6) any combination thereof. In some embodiments, activating Gp140 antibodies bind to the membrane proximal region of Gp140 both before and after tryptic removal of the amino terminal CUB1 domain that has been suggested to mediate dimerization of Gp140. The ability of ActGp140 antibodies to produce one or more of the events 1) through 6) may be measured with respect to any suitable cell type, e.g., keratinocyte cultures.

In some embodiments, the antibodies are “non-activating” Gp140 antibodies (“NactGp140”), in that the binding of these antibodies does not result in one or more of the events 1) through 6) listed above with respect to activating Gp140 antibodies. In some embodiments, binding of non-activating antibodies inhibits the actions of Gp140 agonists (e.g., “blocking” antibodies). The inability of NActGp140 antibodies to produce one or more of the events 1) through 6) and/or the ability of NActGp140 antibodies to inhibit the actions of Gp140 agonists may be measured with respect to any suitable cell type, e.g., keratinocyte cultures.

In some embodiments, Gp140 antibodies are more potent modulators of Gp140 than other modulators, measured by 1) the duration of binding to Gp140 and/or 2) the signal strength measured which is associated with Gp140 binding. For example, though both theActGp140 antibodies described herein and suramin are activators of Gp140, ActGp140 is a more potent activator of Gp140 than suramin, based upon both the duration and signal strength measured upon binding (e.g., in vitro).

In some embodiments antibodies may be coupled to or conjugated to a detectable group or therapeutic group in accordance with known techniques. “Therapeutic group” means any suitable therapeutic group, including but not limited to radionuclides, chemotherapeutic agents and cytotoxic agents (e.g., for cancer treatments).

“Detectable group” as used herein includes any suitable detectable group, such as radiolabels (e.g. ³⁵S, ¹²⁵I, ¹³¹I, etc.), enzyme labels (e.g., horseradish peroxidase, alkaline phosphatese, etc.), fluorescent labels (e.g., fluorescein, green fluorescent protein, etc.), etc., as used in accordance with known techniques.

“Radionuclide” as described herein may be any radionuclide suitable for delivering a therapeutic dosage of radiation to a tumor or cancer cell, including but not limited to ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ^{113m}In, ^{115m}In, ¹²³I, ¹²⁵I, ¹³¹I, ¹⁸⁹I, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, ⁷⁵Se, ¹⁰⁵Ag, ⁸⁹Sr, ³⁵S, ¹⁷⁷Ta, ¹¹⁷mSn, ¹²¹Sn, ¹⁶⁶Yb, ¹⁶⁹Yb, ⁹⁰Y, ²¹²Bi, ¹¹⁹Sb, ¹⁹⁷Hg, 97Ru, ¹⁰⁰Pd, ^{101m}Rh, and ²¹²Pb.

“Chemotherapeutic agent” as used herein includes but is not limited to methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamoxifen, paclitaxel, vincristin, vinblastine, camptothecin, actinomycin D, and cytarabine

“Cytotoxic agent” as used herein includes but is not limited to ricin (or more particularly the ricin A chain), aclacinomycin, diphtheria toxin. Monensin, Verrucarin A, Abrin, Vinca alkaloids, Tricothecenes, and Pseudomonas exotoxin A.

“Subjects” that may be treated by the present invention include both human subjects for medical purposes and animal subjects for veterinary and drug screening and development purposes. Other suitable animal subjects are, in general, mammalian subjects such as primates, bovines, ovines, caprines, porcines, equines, felines, canines, lagomorphs, rodents (e.g., rats and mice), etc. Human subjects are the most preferred. Human subjects include fetal, neonatal, infant, juvenile, adult and geriatric subjects.

“Treat” as used herein refers to any type of treatment or prevention that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc. As such, the term “treatment” also includes prophylactic treatment of the subject to prevent the onset of symptoms. As used herein, “treatment” and “prevention” are not necessarily meant to imply cure or complete abolition of symptoms, but refer to any type of treatment that imparts a benefit to a patient afflicted with a disease, including improvement in the condition of the patient (e.g., in one or more symptoms), delay in the progression of the disease, etc.

“Treatment effective amount”, “amount effective to treat” or the like as used herein means an amount of the agonist or antagonist sufficient to produce a desirable effect upon a patient inflicted with wounds, cancer, tumors, stress, cardiac arrhythmia or other undesirable medical condition in which the regulation of cell junctions by Gp140 is involved. This includes improvement in the condition of the patient (e.g., in one or more symptoms), delay in the progression of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

Formulations and Administration.

For administration in the methods of use described below, the active agent will generally be mixed, prior to administration, with a non-toxic, pharmaceutically acceptable carrier substance (e.g. normal saline or phosphate-buffered saline), and will be administered using any medically appropriate procedure.

The active compounds described above may be formulated for administration in a pharmaceutically acceptable carrier in accordance with known techniques. See, e.g., Remington, The Science And Practice of Pharmacy (9^{th} Ed. 1995). The pharmaceutically acceptable carrier must, of course, also be acceptable in the sense of being compatible with any other ingredients in the composition. The carrier may be a solid or a liquid, or both, and is preferably formulated with the compound as a unit-dose composition, for example, a tablet, which may contain from 0.01 or 0.5% to 95% or 99% by weight of the active compound. One or more active compounds may be incorporated in the compositions of the invention, which may be prepared by any of the well-known techniques of pharmacy comprising admixing the components, optionally including one or more accessory ingredients.

In general, compositions of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

The compositions of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Compositions suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such compositions may be prepared by any suitable method of pharmacy, which includes the step of bringing into association the active compound and a suitable carrier (which may contain one or more accessory ingredients as noted above).

Compositions suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Compositions of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes that render the composition isotonic with the blood of the intended recipient. Aqueous and non-aqueous sterile suspensions may include suspending agents and thickening agents. The compositions may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described.

For example, in one aspect of the present invention, there is provided an injectable, stable, sterile composition comprising an active compound (e.g., zymosan or ActGp140 antibody), or a salt thereof, in a unit dosage form in a sealed container. The compound or salt is provided in the form of a lyophilizate that is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent that is physiologically acceptable may be employed in sufficient quantity to emulsify the compound or salt in an aqueous carrier. One such useful emulsifying agent is phosphatidyl choline.

Compositions suitable for rectal administration are preferably presented as unit dose suppositories. These may be prepared by mixing the active compound with one or more conventional solid carriers, for example, cocoa butter, and then shaping the resulting mixture.

Compositions suitable for topical application to the skin preferably take the form of an ointment, cream, lotion, paste, gel, spray, aerosol, or oil. Carriers that may be used include petroleum jelly, lanoline, polyethylene glycols, alcohols, transdermal enhancers, and combinations of two or more thereof.

Compositions suitable for transdermal administration may be presented as discrete patches adapted to remain in intimate contact with the epidermis of the recipient for a prolonged period of time. Compositions suitable for transdermal administration may also be delivered by iontophoresis (see, for example, Pharmaceutical Research 3 (6):318 (1986)) and typically take the form of an optionally buffered aqueous solution of the active compound. Suitable compositions comprise citrate or bis\tris buffer (pH 6) or ethanol/water and contain from 0.1 to 0.2M of the active ingredient.

Active agents may be provided in lyophylized form in a sterile aseptic container or may be provided in a pharmaceutical formulation in combination with a pharmaceutically acceptable carrier, such as sterile pyrogen-free water or sterile pyrogen-free physiological saline solution.

Dosage of the active agent for the methods of use described below will depend, among other things, the condition of the subject, the particular condition being treated, the route of administration, the nature of the therapeutic agent employed, and the sensitivity of the tumor to the particular therapeutic agent. For example, in some embodiments, the dosage is about 1 to 10 micrograms per kilogram subject body weight. The specific dosage of the active agent is not critical, as long as it is effective to result in some beneficial effects in some individuals within an affected population. In general, the dosage may be as low as about 0.05, 0.1, 0.5, 1, 5, 10, 20 or 50 micrograms per kilogram subject body weight, or lower, and as high as about 5, 10, 20, 50, 75 or 100 micrograms per kilogram subject body weight, or even higher.

Methods of Treatment.

In general, Gp140 is a cellular response mechanism, acting as a reporter and/or responder to various types of stress in the body, detecting disruption to tissue or organ integrity (e.g., chemical or mechanical integrity, viral insult, etc.). As used herein, “stress” refers to the exposure of a cell, a tissue or organ to an insult, injury or other detrimental event to which the body must normally respond in order to regain or maintain health and/or optimal function. For example, stress as used herein may refer to a wound of the skin, a cardiac or neuronal insult, an inflammatory reaction or condition, etc. Without wishing to be bound by theory, this role of Gp140 is also consistent with the theory of cancer as a response of the body to chronic stress and inflammation.

Accordingly, in some embodiments of the present invention, active agents (e.g., Gp140 agonists or antagonists) are used to treat cell, tissue or organ stress in a subject, including, but not limited to, treatment of wounds, cancer treatment, treatment for an inflammatory response or disorder, treatment for a cardiac disease or insult, and treatment for a neuronal disease or insult.

### A. Methods of Use: Wound Healing.

An aspect of the present invention is a method of treating a wound (e.g., burns, abrasions, lacerations, incisions, pressure sores, puncture wounds, penetration wounds, gunshot wounds, crushing injuries, etc.) in a subject in need thereof, by administering an active agent as described herein (e.g., formulated in a cream or ointment for administration) to the wound in an amount effective to treat the wound (i.e., promote healing). Wounds may be acute or chronic.

“Acute” wounds are those without an underlying deficit in healing, which heal in an orderly set of stages and in under 3 months (e.g., cuts or scrapes): A “chronic” wound is a wound that does not heal in an orderly set of stages and/or does not heal in 3 months or less (e.g., venous ulcers, diabetic ulcers and pressure ulcers).

Microbes are a ubiquitous component of epidermal wounds and are a major cause of chronic wounds. Wounding of quiescent epidermis must disassemble cell-cell junctions in order to permit migration of leading keratinocytes at the wound edge but must also assemble new cell-cell junctions to exclude microbes.

Yeast and bacteria are ubiquitous inhabitants of epidermis but do not present a health risk unless the epithelium is wounded and/or immunodeficiency exists. In wounds, the pathogens activate innate immunity by interacting with three known defense systems, complement, scavenger receptors and Toll-like receptors. Further, the epidermis provides a barrier function that excludes microbes even before completion of wound closure: Migratory keratinocytes can assemble tight junctions to exclude microbes even in the absence of the granular cell layer that provides the barrier function in the intact epidermis.

Wounding of quiescent epidermis activates polarized migration of leading and following keratinocytes. Cx43 is normally downregulated in leading keratinocytes at the wound edge in the first 24-48 hours, and wound repair is accelerated if this downregulation is enhanced.

Quiescent basal keratinocytes in epidermis adhere to laminin 332/5 in the basement membrane via integrin α6b4 in hemidesmosome (HD) cell junctions, adhere to each other via cadherins in adherens junctions (AJs), occludin, claudins and JAMs in tight junctions (TJs) (Miyoshi et al., Adv Drug Deliv Rev 57:815-55, 2005) and communicate with each other via Cx43 in gap junctions (GJs) (Lampe et al., Journal of Cell Biology 143:1735-47, 1998). Wounding disrupts the cell junctions to generate an epidermal outgrowth composed of migratory leading keratinocytes and confluent following keratinocytes. Leading keratinocytes down-regulate, HDs, AJs, TJs and GJs but increase cell migration via β₁ integrins over exposed dermal collagen and fibronectin (Lampe et al., Journal of Cell Biology 143:1735-47, 1998; Nguyen et al., Curr. Opin. Cell Biol. 12:554-562, 2000; Mertens et al., Trends Cell Biol 16: 308-16, 2006). Following cells maintain intercellular communication via GJs and intercellular adhesion via AJs and TJs. Leading and following keratinocytes are distinguished based on expression of repair components in the wound outgrowth (Harper et al., J. Cell Sci. 118:3471-3485, 2005). Leading cells utilize β1 integrins for migration via Rho-dependent mechanisms over dermal collagen and fibronectin (Nguyen et al., Curr. Opin. Cell Biol. 12:554-562, 2000), express and deposit precursor laminin 5 and disassemble GJs. In contrast, following cells communicate via GJs and migrate over deposits of laminin 5 via α3β₁ in a PI3K and/or Rac-dependent mechanism.

Consistently, adhesion of following cells to deposited laminin 5 via α3β1 and α6β4 promotes cell-cell adhesion via cadherins and communication via GJs while adhesion to dermal components via β1 integrins disrupts intercellular interactions in leading cells (Lampe et al., Journal of Cell Biology 143:1735-47, 1998). Tiam 1 is a GTP exchange factor (GEF) and activator of Rac that regulates both integrin-substrate interactions in leading cells and apical-basal cell polarization, and assembly of primordial adhesions as precursors to both AJs and TJs in following cells (Mertens et al., Trends Cell Biol 16: 308-16, 2006). Consistently, Tiam 1-deficeint keratinocytes are defective in deposition of laminin 332/5 owing to impaired α3β1-integrin-induced Rac activation (Hamelers et al., J Cell Biol 171:871-81, 2005). Consistently, α3β1 interactions with laminin 332 activates Rac on the substratum that stabilizes the lamellipodium (Choma et al., J Invest Dermatol 127:31-40, 2007; Choma et al., J Cell Sci 117:3947-59, 2004) and polarized persistent migration (Frank et al., J. Cell Sci. 117:1351-1363, 2004).

Cx43 regulation plays an important role in epidermal function and wound repair. It has been suggested that GJC may regulate certain aspects of the wound healing including synchronization of cellular migration. Both rat and human skin show decreased connexin expression at the wound edge and rat skin shows variable expression of different connexins in areas proximal to the wound with a return to homeostatic levels upon wound closure (Saitoh et al., Carcinogenesis 18:1319-1328, 1997). Cx43 antisense application to wounds accelerates keratinocyte migration and the rate of wound repair resulting in less scaring (Qiu et al., Curr. Biol. 13:1697-703, 2003; Kretz et al., Journal of Cell Science 116:3443-52, 2003). We have shown that human keratinocytes require GJC to initiate migration (Richards et al., Journal of Cell Biology 167:555-62, 2004).

Wound closure can be particularly slow in diabetic subjects or in elderly subjects, which may result in chronic wounds that can easily become infected. Abnormal connexin expression has been shown to underlie delayed wound healing in the skin of diabetic subjects (Wang et al., “Abnormal connexin expression underlies delayed wound healing in diabetic skin,” Diabetes 56: 28-9-2817, 2007). Preventing the upregulation of Cx43 in diabetic wounds significantly improved the rate of healing, demonstrating the therapeutic value of Cx43 targeting in such wounds. In addition, promotion of the barrier function of wound healing through the formation of tight juctions would be beneficial.

We have found that both Cx43 and α-catenin are regulated by the activation of Gp140, and therefore Gp140 is an attractive target for cell junction modulation in wounds. Examples of active agents useful for treatment of wounds according to the present invention include, but are not limited to, agonists such as activating antibodies, zymosan, Staphylococcus aureus peptidoglycan, and suramin. In other embodiments, wounds may be treated with antagonists such as non-activating Gp140 antibodies or siRNA targeting Gp140 either directly or indirectly.

### B. Methods of Use: Cancer Treatments.

Another use of agonists or antagonists is to treat cancers or tumors, particularly those that have Gp140 receptors (e.g., colon cancer, lung cancer, prostate cancer, breast cancer, skin cancer such as malignant or metastatic melanoma, etc.). Several studies have indicated that the loss of gap junctional communication contributes to carcinogenesis in several cell types, including mammary epithelial cells, and agents that restore gap junctional communication act as tumor suppressors (see, e.g., Hirschi et al., Cell Growth & Differentiation 7:861-870, 1996). For example, the loss of Cx43 gap junction in breast tumor cells is a critical step in carcinogenesis, and restoration of gap junctional communication may be useful in breast cancer treatment, for example, by increasing the bystander effects of other therapies through renewed intercellular communication (Laird et al., “Deficiency of connexin43 gap junctions is a independent marker for breast tumors,” Cancer Res. 59: 4104-4110, 1999). The small proportion of molecules targeted by antibody-delivered toxins or by chemotherapeutics could more effectively spread to adjacent cells through gap junctional communication, which allows improved efficacy through the bystander effect.

We have found that both Cx43 and α-catenin are regulated by Gp140, and therefore Gp140 is an attractive target for cell junction modulation in methods of treatment for cancer by promoting gap junctional communication and/or cell-cell adhesion. Examples of active agents useful for treatment according to the present invention include, but are not limited to, agonists such as activating antibodies, zymosan, Staphylococcus aureus peptidoglycan, and suramin. In other embodiments, cancer may be treated with antagonists such as non-activating Gp140 antibodies or siRNA targeting Gp140 either directly or indirectly.

In the treatment of cancers or tumors the active agents of the present invention may optionally be administered in conjunction with other, different, cytotoxic agents such as chemotherapeutic or antineoplastic compounds or radiation therapy useful in the treatment of the disorders or conditions described herein (e.g., chemotherapeutics or antineoplastic compounds). The other compounds may be administered concurrently. As used herein, the word “concurrently” means sufficiently close in time to produce a combined effect (that is, concurrently may be simultaneously, or it may be two or more administrations occurring before or after each other) As used herein, the phrase “radiation therapy” includes, but is not limited to, x-rays or gamma rays which are delivered from either an externally applied source such as a beam or by implantation of small radioactive sources.

Suramin is known to inhibit the growth of various types of cancer, e.g., osteogenic sarcoma, endometrial, breast, ovarian, lung, prostate, lymphoma and skin (malignant melanoma) (Eisenberger et al., “The experience with suramin in advanced prostate cancer,” Cancer 75(S7):1927-1934, 1994). Suramin has been in clinical trials for the treatment of hormone-refractory prostate cancer, alone and in combination with other cytotoxic agents (e.g., 5-fluorouracil, topotecan (Hycamptin®, a topoisomerate inhibitor marketed by GlaxoSmithKline), etc.). However, there have been reports of renal toxicity associated with suramin treatment (Figg et al., Cancer 74(5):1612-1614, 1994). Therefore, in some embodiments, more potent and/or specific agonists or antagonists of Gp140 are used in the methods of treatment, e.g., activating Gp140 antibodies, siRNA, etc.

Examples of other suitable chemotherapeutic agents which may be concurrently administered with active agents as described herein include, but are not limited to, Alkylating agents (including, without limitation, nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide; Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine; Natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol®), Mithramycin, Deoxyco-formycin, Mitomycin-C, L-Asparaginase, Interferons (especially IFN-a), Etoposide, and Teniposide; Other anti-proliferative cytotoxic agents are navelbene, CPT-11, anastrazole, letrazole, capecitabine, reloxafine, cyclophosphamide, ifosamide, and droloxafine.

Additional anti-proliferative cytotoxic agents include, but are not limited to, melphalan, hexamethyl melamine, thiotepa, cytarabin, idatrexate, trimetrexate, dacarbazine, L-asparaginase, camptothecin, topotecan, bicalutamide, flutamide, leuprolide, pyridobenzoindole derivatives, interferons, and interleukins. Preferred classes of antiproliferative cytotoxic agents are the EGFR inhibitors, Her-2 inhibitors, CDK inhibitors, and Herceptin® (trastuzumab). (see, e.g., U.S. Pat. No. 6,537,988; U.S. Pat. No. 6,420,377). Such compounds may be given in accordance with techniques currently known for the administration thereof.

See also U.S. Pat. No. 6,599,912 to Au et, al. for additional cytotoxic agents.

### C. Methods of Use: Inflammatory Disease.

A further use of agonists or antagonists is to treat inflammatory disease, e.g., cystic fibrosis. Without wishing to be bound by theory, inhibition of gap junctional communication is predicted to restrict the passage of inflammatory activators, thereby containing the spread of the inflammatory response (see, e.g., Brosnan et al., Am. J. Pathology 158(5):1565-1569, 2001).

Active agents useful for treatment of inflammatory disease according to the present invention include, but are not limited to, agonists such as activating antibodies, zymosan, Staphylococcus aureus peptidoglycan, and suramin. In other embodiments, antagonists are used, such as non-activating Gp140 antibodies or siRNA targeting Gp140, either directly or indirectly.

### D. Methods of Use: Cardiac Function/Disease.

A further use of agonists or antagonists is in the treatment of cardiac disease. Promoting gap junctional communication has been shown to attenuate the risk of cardiac arrhythmias (see, e.g., Amino et al., “Heavy ion radiation up-regulates Cx43 and ameliorates arrhythmogenic substrates in hearts after myocardial infarction,” Cariovascular Res. 72: 412-421, 2006).

Studies have also demonstrated that transient inhibition of gap junctional communication during myocardial reperfusion limits infarct size when used in concentrations that only minimally affected myocardium electrical impedence (see, e.g., Rodriguez-Sinovas et al., “Enhanced effect of gap junction uncouplers on macroscopic electrical properties of reperfused myocardiam,” J. Physiol 559.1: 245-257, 2004). In addition, inhibition of gap junctional communication prior to ischemia preserves the electrical coupling of cells and is anti-arrhythmic during a subsequent ischemic insult (see Papp et al., “Gap junctional uncoupling plays a trigger role in the antiarrhythmic effect of ischaemic preconditioning,” Cardiovascular Res. 74: 396-405, 2007).

A loss of the Cx43 protein has also been associated with the age-related deterioration of the cardiac pacemaker (see, e.g., Jones et al., “Ageing-related changes of connexins and conduction within the sinoatrial node,” J. Physiol 560.2: 429-437, 2004).

We have found that Cx43 is regulated by Gp140, and therefore Gp140 is an attractive target for cell junction modulation in cardiac treatments. Examples of active agents useful for treatment of cardiac function/disease according to the present invention include, but are not limited to, agonists such as activating antibodies, zymosan, Staphylococcus aureus peptidoglycan, and suramin. In other embodiments, cardiac function/disease may be treated with antagonists such as non-activating Gp140 antibodies or siRNA targeting Gp140 either directly or indirectly.

### E. Methods of Use: Brain Trauma/Injury.

A still further use of agonists or antagonists is to treat brain trauma or injury. Studies have demonstrated that gap junctional communication enhances neural tissue vunerability to traumatic injury, and simultaneous knockdown of two neuronal connexins resulted in significant neuroprotection (see, e.g., Frantseva et al., J. Neurosci. 22(3):644-653, 2002; Rawanduzy et al., “Effective reduction of infarct volume by gap junction blockade in a rodent model of stroke,” J. Neurosurg. 87(6): 916-920, 1997).

We have found that Cx43 is regulated by Gp140, and therefore Gp140 is an attractive target for cell junction modulation in brain trauma or injury. Examples of active agents useful for treatment of brain trauma or injury according to the present invention include, but are not limited to, agonists such as activating antibodies, zymosan, Staphylococcus aureus peptidoglycan, and suramin. In other embodiments, brain trauma/injury may be treated with antagonists such as non-activating Gp140 antibodies or siRNA targeting Gp140 either directly or indirectly.

Preparation of Antibodies.

Antibodies and the production thereof are known. See, e.g., U.S. Pat. No. 6,849,719; see also U.S. Pat. Nos. 6,838,282; 6,835,817; 6,824,989. In some embodiments of the present invention, the antibodies are “Gp140” antibodies, in that they specifically bind to Gp140, as measured by the ability of the antibodies to immunoprecipitate the Gp140 protein.

In some embodiments, antibodies are “activating” Gp140 antibodies (“ActGp140”), in that they activate the actions of Gp140. For example, in some embodiments the antibodies are monoclonal (e.g., mouse monoclonal) antibodies, which may be prepared as described below. n some embodiments hybridomas are selected based upon the ability of the antibodies produced to: 1) promote the assembly of Gp140 membrane clusters; 2) activate SFKs and increases phosphorylation of Gp140, SFK and PKCδ by SFK; 3) recruit PKCδ to the detergent-resistant Gp140 membrane cluster as a complex with Gp140; 4) promote assembly of GJs and/or tight junction proteins; 5) increase phosphorylation of Cx43 at S279/282 by MAPK, and/or at 5368 by PKC and/or Y247/265 by SFK; or 6) any combination thereof. The ability of ActGp140 antibodies to produce one or more of the events 1) through 6) may be measured with respect to any suitable cell type, e.g., keratinocyte cultures. In some embodiments, activating Gp140 antibodies bind to the membrane proximal region of Gp140 both before and after tryptic removal of the amino terminal CUB1 domain that has been suggested to mediate dimerization of Gp140.

In other embodiments, the antibodies are “non-activating” Gp140 antibodies

(“NactGp140”). For example, in some embodiments the antibodies are monoclonal (e.g., mouse monoclonal) antibodies, which may be prepared as described below. In some embodiments hybridomas are selected that do not result in one or more of the events 1) through 6) listed above with respect to activating Gp140 antibodies. In further embodiments, hybridomas are selected based upon the ability of the antibodies produced to inhibit the actions of Gp140 agonists (e.g., “blocking” antibodies). The inability of NActGp140 antibodies to produce one or more of the events 1) through 6) and/or the ability of NActGp1140 antibodies to inhibit the actions of Gp140 agonists may be measured with respect to any suitable cell type, e.g., keratinocyte cultures.

Antibodies of the invention include antibodies that are modified, i.e., by the covalent attachment of any type of molecule to the antibody such that covalent attachment does not prevent the antibody from specifically binding to its binding site. For example, antibodies of the invention may be modified, e.g., by glycosylation, acetylation, pegylation, phosphylation, amidation, or with other protecting/blocking groups, proteolytic cleavage, linkage to a cellular ligand or other protein, etc. Any of numerous chemical modifications may be carried out by known techniques, including but not limited to specific chemical cleavage, acetylation, formylation, metabolic synthesis of tunicamycin, etc. Additionally, the antibodies may contain one or more non-classical amino acids.

Polyclonal antibodies of the invention can be generated by any suitable method known in the art. For example, a suitable antigen can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

Monoclonal antibodies can be prepared using a wide variety of techniques including the use of hybridoma, recombinant, and phage display technologies, or a combination thereof. For example, monoclonal antibodies can be produced using hybridoma techniques including those taught, for example, in Harlow et al., Antibodies: A Laboraₜory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and known. Briefly, mice are immunized with an antigen or a cell expressing such antigen. Once an immune response is detected, e.g., antibodies specific for the antigen are detected in the mouse serum, the mouse spleen is harvested and splenocytes isolated. The splenocytes are then fused by known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments that recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)₂ fragments). F(ab′)2 fragments contain the variable region, the light chain constant region and the CHI domain of the heavy chain.

For example, antibodies can also be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include but are not limited to those disclosed in U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art.

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and. a human immunoglobulin constant region. Methods for producing chimeric antibodies are known in the art. See e.g., Morrison, Science 229:1202 (1985); Oi et al., BioTechniques 4:2.14 (1986); Gillies et al., (1989) J. Immunol. Methods 125:191-202; U.S. Pat. Nos. 5,807,715; 4,816,567; and 4,816,397, which are incorporated herein by reference in their entireties. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (see, e.g., U.S. Pat. Nos. 5,225,539; 5,530,101; and 5,585,089), veneering or resurfacing (see, e.g., EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are desirable for therapeutic treatment, diagnosis, and/or detection of human patients. Human antibodies can be made by a variety of methods known in the art including phage display methods described above using antibody libraries derived from human immunoglobulin sequences. See, e.g., U.S. Pat. Nos. 4,444,887 and 4,716,111.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,413,923; 5,625,126; 5,633,425; 5,569,825; 5,661,016; 5,545,806; 5,814,318; and 5,939,598.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

The invention further provides polynucleotides comprising a nucleotide sequence encoding an antibody of the invention as described above. The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligation of those oligonucleotides, and then amplification of the ligated oligonucleotides by PCR. Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may bₑ obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by

PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Further discussion of antibodies with respect to Gp140 can be found in U.S. Patent Application No. 20070031419 to Domon et al.

### Implants.

Active compounds of the invention, particularly antagonists such as antibodies or peptides as described above, may be coupled to or conjugated to implants or implantable medical devices in accordance with known techniques for carrying out the methods described herein, or for combating problems associated with the implant such as stenosis and restenosis. See, e.g., U.S. Pat. Nos. 6,786,922; 6,746,686; 6,718,208; 6,617,142; 6,352,832; 6,238,872. Any implant can be so utilized, including but not limited to stents (e.g., vascular stents), electrodes, catheters, leads, implantable pacemaker or cardioverter housings, joints, screws, rods, ophthalmic implants (including, but not limited to, intraocular lens implants, glaucoma implants or drainage implants, and punctal implants or plugs), etc. The implants may be of any suitable material, including but not limited to organic polymers (including stable or inert polymers and biodegradable polymers), metals such as stainless steel and titanium, inorganic materials such as silicon, and composites thereof.

### EXAMPLES

### Example 1

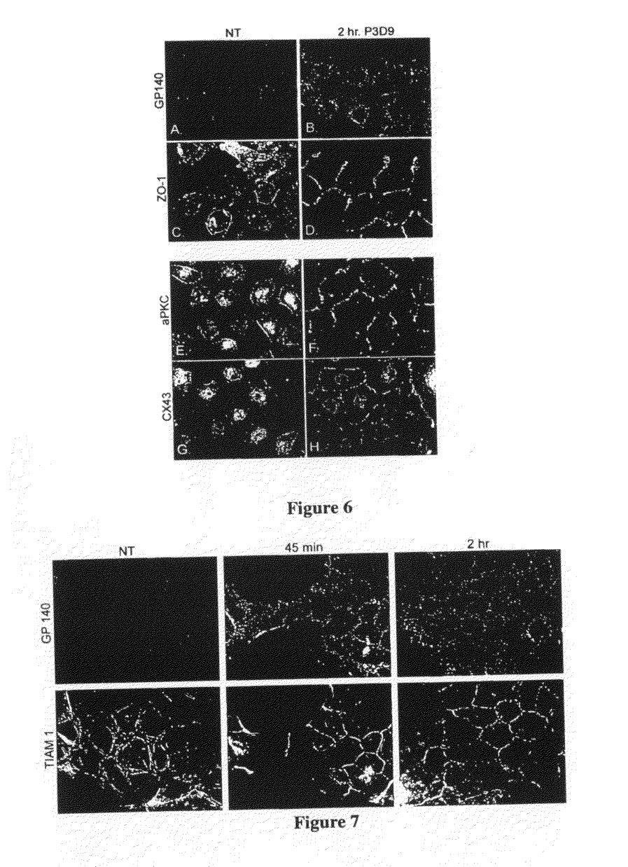

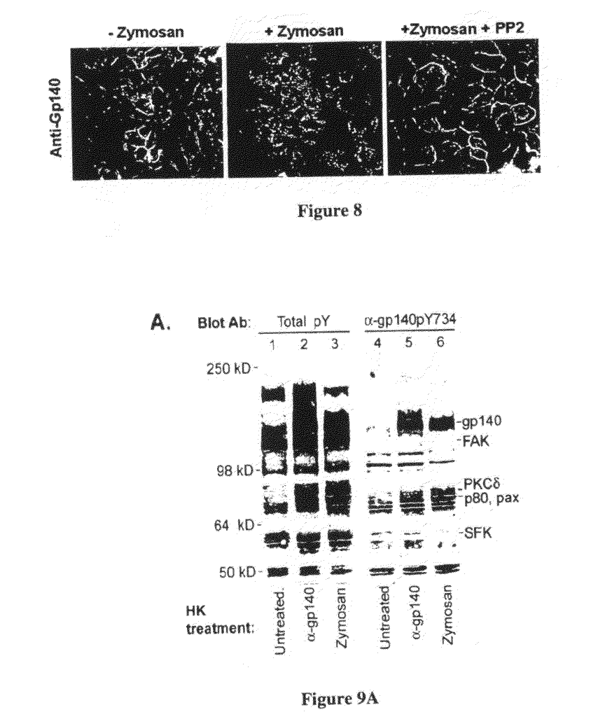

Signals through Gp140 in cell-cell contacts are sufficient to promote gap junction assembly. To evaluate the possible role of Gp140 as a promoter of GJs, we cultured keratinocytes under conditions to reduce SFK activity and cell-cell interactions (30 μM Ca⁺²) (Xie et al., “Calcium-induced human keratinocyte differentiation requires src- and fyn-mediated phosphatidylinositol 3-kinase-dependent activation of phospholipase C-gamma1,” Mol Biol Cell 16: 3236-46, 2005) that are used for studies of tight junction assembly (Mertens et al., “The Rac activator Tiam1 controls tight junction biogenesis in keratinocytes through binding to and activation of the Par polarity complex,” J Cell Biol 170: 1029-37, 2005). Significantly, contact of zymosan with keratinocytes (FIG. 5) or binding of ActGp140 mAb, but not the NActGp140 mAb, to keratinocytes (FIG. 6) led to assembly of Gp 140 into membrane clusters in cell-cell contacts and to the rapid and dramatic recruitment of components of GJs and tight junctions into the cell-cell contacts. The recruited components included ZO-1, afadin (AF-6) (shown in FIG. 5), Cx43 and ZO-2 and the PAR polarity complex including Par 3 and atypical PKC (aPKC/PKCζ) (Miyoshi et al., “Molecular perspective on tight junction assembly and epithelial polarity,” Adv Drug Deliv Rev 57:815-55, 2005). The recruitment was apparent within 30 min of addition of zymosan or ActGp140 mAb and correlated with the assembly of the Gp140 membrane cluster (FIG. 3). Significantly, Gp140 was internalized after 2 h of treatment while the junctional components were retained in cell-cell contacts (FIG. 6). This suggested that signals through Gp140 function as a catalyst that is sufficient to assemble cell-cell junctions but was not required for maintaining GJ or TJs.

In controls, we did not detect E-cadherin, p120 catenin, α-catenin or β-catenin, FAK, or vinculin in cell-cell contacts in response to zymosan or ActGp140 mAb. This suggested that contact of zymosan or ActGp140 mAbs can lead to assembly of Gp140 membrane clusters and selectively recruit Cx43 into cell-cell contacts in the absence of E cadherin. Further, this suggests that signals through Gp140 may be a physiologically significant regulator of cell-cell junctional assembly in response to microbes as a mechanism to exclude microbes from wounds. Further studies can evaluate recruitment of adhesion receptors (JAMs, claudins, occludins) known to assemble in initial “primordial adhesions” that precedes assembly of tight and adherens junctions. The results suggest that zymosan may signal through Gp140 to promote assembly of tight junctions as a physiological response in host defense. It also raises the possibility that Gp140 may promote assembly of primordial adhesions as precursors to tight junctions and possibly gap junctions.

To establish that zymosan and ActGp140 mAbs led to formation of functional GJs, we measured GJC in the presence of ActGp140 mAb (P3D9) or NActGp140 (P5H10) or control. The ActGp140 mAb recruited Cx43 into detergent-resistant clusters in cell-cell contacts and increased GJC as measured by a ˜3× increase in the length of dye spread (Control=26.1±4.8 μm; Act mAb=73.6±20.2 μm; NAc Mab=22.6±4.6 μm; Act Mab different from the others, p<0.015).

### Example 2

Microbes and anti-Gp140 mAbs signal through Rac-Tam 1 to assemble GJs. Our results establish that interaction of extracellular Gp140 in cell-cell contacts with zymosan or ActGp140 mAb is sufficient to assemble Cx43 into functional GJs. The signals through Gp140 also recruited components of the Par polarity complex (including aPKC, Par3). At this time, it is not known if the Par polarity complex interacts with Cx43 or other membrane components. Gp 140 does not have a known PDZ domain (Ranganathan et al., “PDZ domain proteins: Scaffolds for signaling complexes,” Current Biol. 7: R770-R773, 1997), so it is unlikely that ZO-1, ZO-2 or Par3 interact with Gp140 directly. Therefore, we hypothesized that signals through Gp140 recruit and activate a regulator of the Par polarity complex. Studies by others have established that the Par polarity complex is regulated by Rac GTPase, a regulator of the actin cytoskeleton, and by Rac GTP exchange factors including Tiam 1 (see Mertens et al. (2006) Trends Cell Biol. 16:308-316). Consistently, Rac and Tiam 1 are required for wound closure and for polarization of leading and following keratinocytes (Nguyen et al., “Deposition of laminin 5 in epidermal woulds regulates integrin signaling and adhesion,” Curr. Opin. Cell Biol. 12:554-562, 2000; Mertens et al., “Tiam1 takes PARt in cell polarity,” Trends Cell Biol 16: 308-16, 2006). With this in mind, we found that ligation of Gp140 with ActGp140 rapidly recruits Tiam 1 (FIG. 7) and Racl (results not shown) to cell-cell contacts. Further, treatment of keratinocytes with NSC23766, a reversible inhibitor of Rac (Gao et al., “Rational design and characterization of a Rac GTPase-specific small molecule inhibitor,” Proc Natl Acad Sci USA 101: 7618-23, 2004) prevents maturation of primordial adhesions into tight junctions, using ZO-1 as a reporter, for binding of ActGp140 mAb.

We conclude that ligation of Gp140 with ActGp140 mAbs in cell-cell contacts of keratinocytes assembles functional GJs and tight junctions possibly by activating Rac-Tiam 1 signaling. We hypothesize that microbes will function similar to the Act Gp140 mAb, and that signals through Gp140 function to exclude microbes in epithelial cells and this function will be critical to the barrier function of keratinocytes in the migratory outgrowth in wounds.

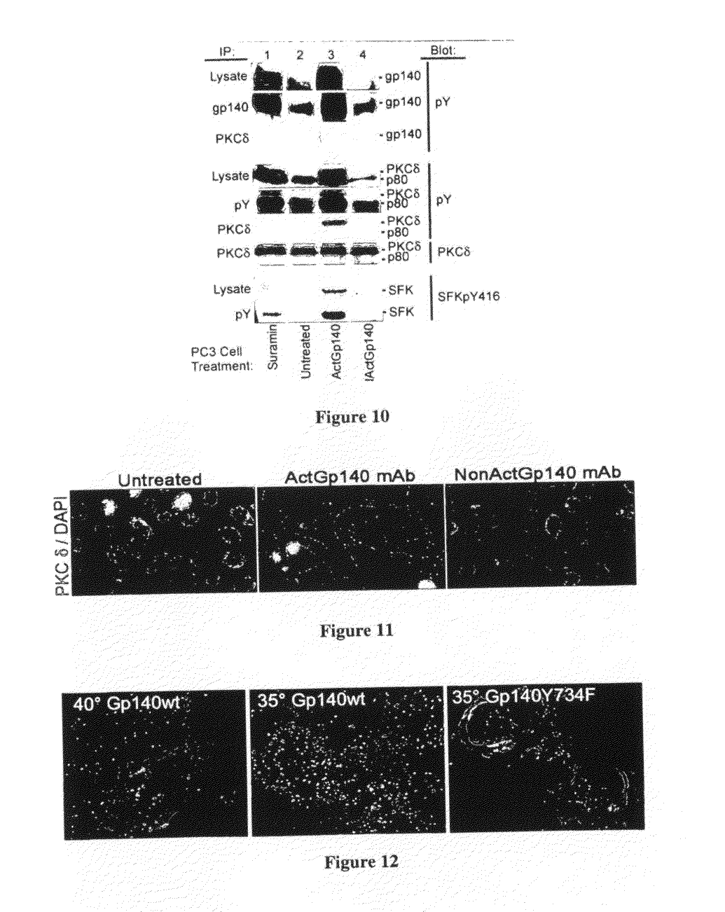

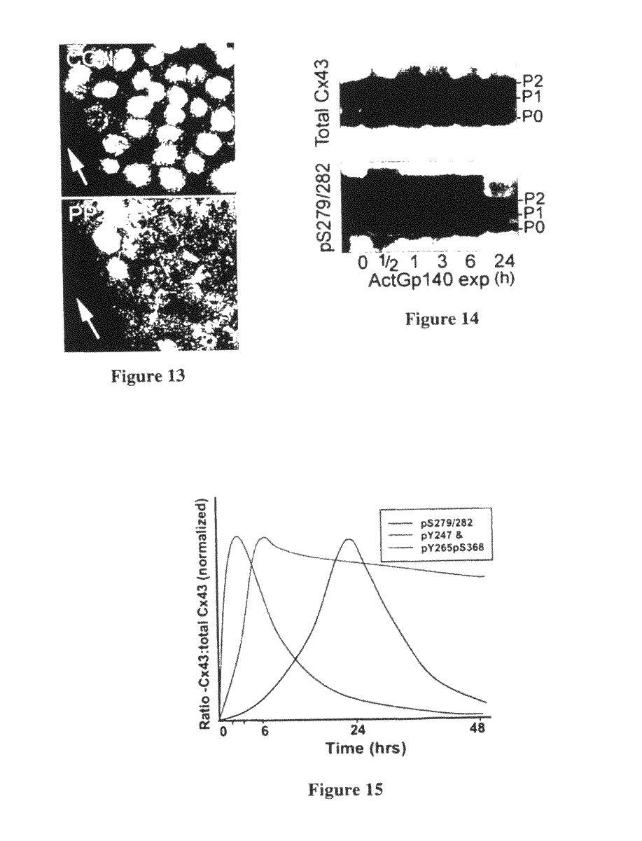

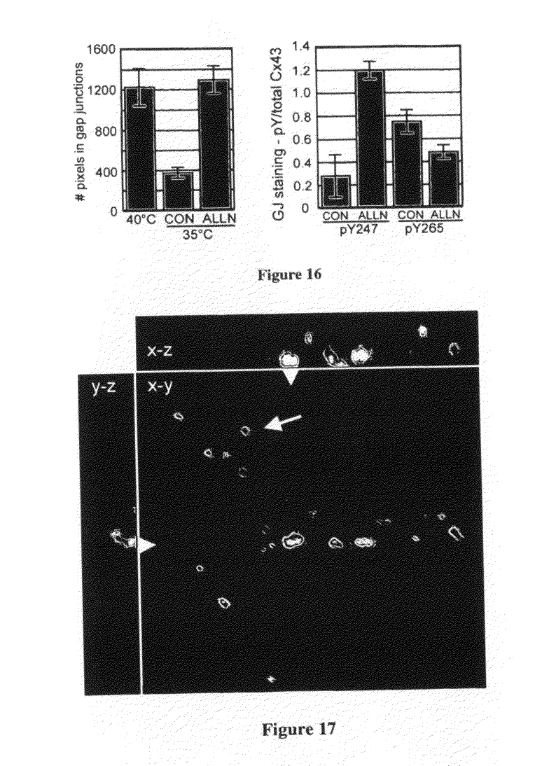

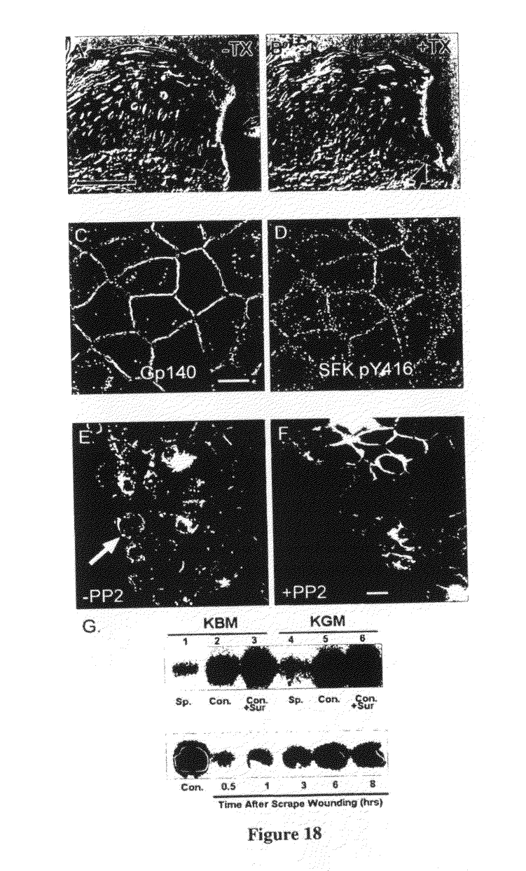

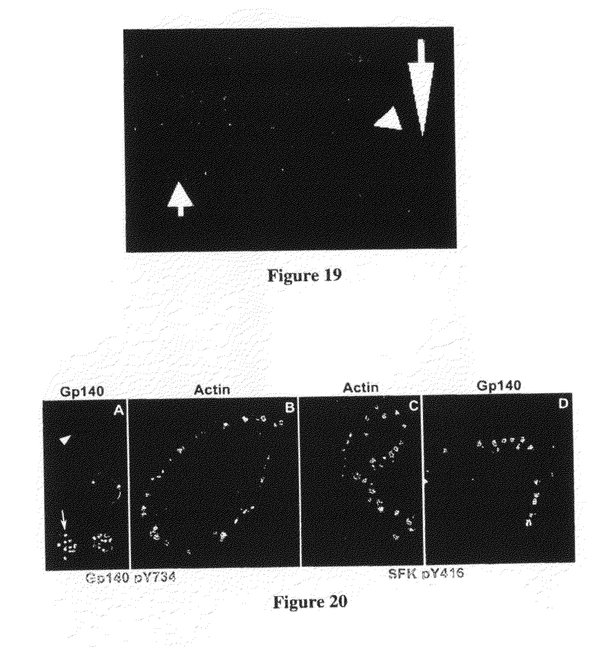

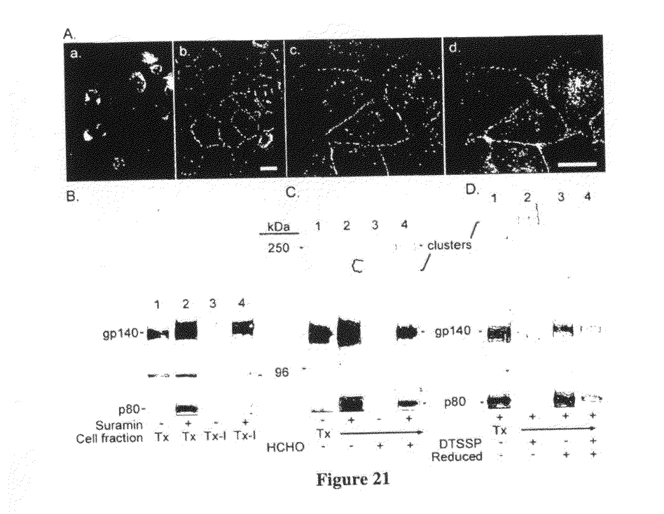

We hypothesize that the extracellular CUB domains of Gp140 participate in the assembly of the Gp140 membrane cluster that recruits the signaling and junctional components. The extracellular CUB domains of Gp140 are homologous to CUB1 dimerization domains of MASP and C1r components of the complement cascade. This suggests that CUB1 of Gp140 may mediate homodimerization in cis and/or trans configuration to assemble Gp140 membrane clusters in cell-cell contacts. Consistently, the NActGp140 that binds CUB1 (FIG. 10), can inhibit phosphorylation of Gp140 induced by the ActGp140 mAb. Further, protease digestion of Gp140 that removes CUB1 prevents phosphorylation.

### Example 3

### Characterization of the Microbe-Induced Gp140 Membrane Clusters in Cell-Cell Contacts at the Light and Ultrastructural Levels

A. Characterization of the Gp140 membrane clusters induced by zymosan. We asked if assembly of Gp140 into membrane clusters was restricted to sites of contact with zymosan particles or ActGp140 mAb. To answer this question, we immobilized ActGp140 mAb on beads, centrifuged the beads onto the apical surface of adherent keratinocytes, washed to remove unbound beads, fixed and permeabilized cells (FIG. 20).

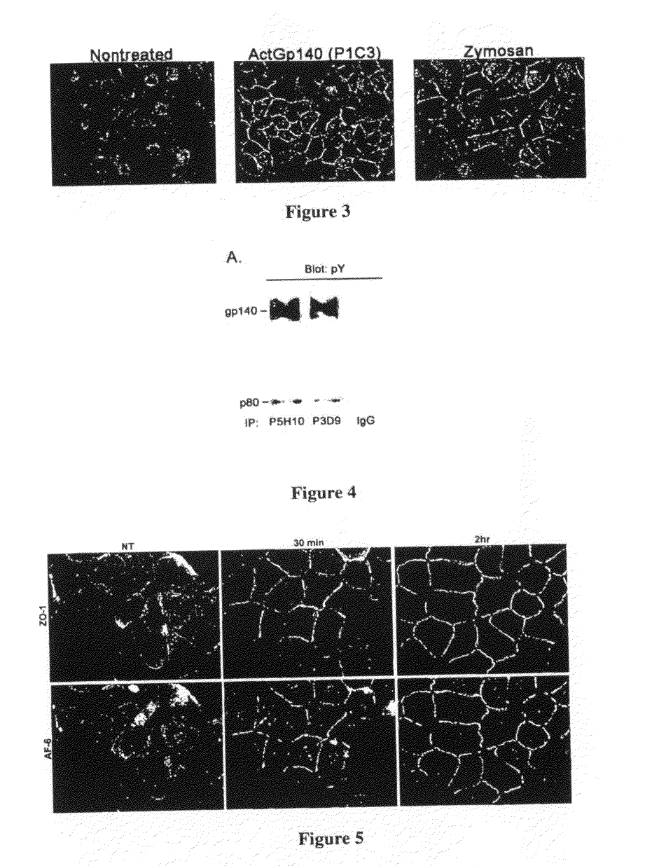

B. Evaluate the activating and inhibitory roles of ActG140 and NactGp140 Mab Fabs in regulating signals through Gp140. We have generated mouse mAbs against different epitopes in the extracellular domain of human Gp140 for use in analysis of the structure-function of Gp140. Some of the mAbs have been characterized and grouped as either ActGp140 mAbs (e.g., clones P3D9, P1C3, P4E8) and NactGp140 mAbs (e.g., clones P5H10, P3B5) that all immunoprecipitate Gp140 (FIG. 4). Binding of ActGp140 mAb to keratinocytes duplicates addition of zymosan in: (a) assembling Gp140 membrane clusters, (b) activating SFKs, and increasing phosphorylation of Gp140, SFK and PKCδ by SFK, (c) recruiting PKCδ to the detergent-resistant Gp140 membrane cluster as a complex with Gp140, (d) promoting assembly of GJs and tight junction proteins, (e) increasing phosphorylation of Cx43 at S279/282 by MAPK, at S368 by PKC and Y247/265 by SFK. ActGp140 mAbs bind to the membrane proximal region of Gp140 both before and after tryptic removal of the amino terminal CUB1 domain that has been suggested to mediate dimerization of Gp140. In contrast, NactGpl 40 do not activate functions a-e and our recent studies indicate that at least some of these NactGp140 mAbs inhibit these functions possibly by binding to CUB1 of Gp140 and preventing the dimerization induced by the ActGp140 mAbs.

Fab fragments of ActGp140 mAbs and NactGp140 mAbs are prepared to determine if monovalent Fabs can promote phosphorylation of Gp140 without the possibility of cross-linking. From this it is concluded whether ligation of Gp140 CUB domains with monovalent Fab ActGp140 activates dimerization of Gp140 leading to phosphorylation. If monovalent Fabs bind to Gp140 but do not promote phosphorylation, it is concluded that Mab cross-linking is required for phosphorylation. To confirm this point, we multimerize the Fab ActGp140 on surfaces to establish the requirement for multimeric interactions. If Fab NactGp140 mAbs inhibit signaling by ActGp140 mAbs or zymosan, we will map the epitopes recognized by both the ActGp140 and NactGp140 mAbs using either libraries of synthetic peptides or domain swaps with mouse Gp 140, that do not react with any of the mAbs listed.

### Example 4

### Expression of Soluble Forms of Gp140 CUB1 and Evaluation of Their Effects on Assembly of Clusters and Signaling Through Gp140

A. Preparation of recombinant extracellular Gp140 (rExoGp140) and recombinant CUB domains (rCubGp140): A full-length Gp140/CDCP1 cDNA was constructed as previously described (Brown et al., “Adhesion or plasmin regulates tyrosine phosphorylation of a novel membrane glycoprotein p80/gp140/CUB domain-containing protein 1 in epithelia,” J. Biol. Chem. 279: 14772-83, 2004) and cleaved with AIM and HindIII restriction enzymes and the resulting ends were blunted with T4 DNA Polymerase to generate a ExoGp140 cDNA. The ExoGp140 cDNA (nucleotides 1-1989 starting from the initialing ATG, amino acids 1-663) was inserted into a blunt ended fusion expression cassette in frame with murine IgG2a Fc in a pcDNA3.1 vector. The resulting open reading frame encoded a fusion protein consisting of N-terminal ExoGp140 and C-terminal murine IgG2aFC. We generate a restriction fragment of ExoGp140 cDNA in order to prepare cDNA for CUB1 of Gp140 (CublGp140 cDNA). Alternatively, we prepare recombinant CUB1 domain as a GST fusion protein expressed in bacteria for dimerization studies as previously reported for the CUB1 domains of MASPs (Thielens et al., “Interaction properties of human mannan-binding lectin (MBL)-associated serine proteases-1 and -2, MBL-associated protein 19, and MBL,” J Immunol 166: 5068-77, 2001).

ExoGp140 was expressed in NSO mouse myeloma cells or 293T cells and secreted as a 140 kDa fusion protein under reducing conditions and as a dimer of 280 kDa under non-reducing conditions (results not shown because of space limits). Both the monomer and dimer bind anti-IgG2a antibody and with a Mab prepared against recombinant ExoGp140 (rExoGp 140).

B. Binding of soluble recombinant rExoGp140 to cell. PC3 human prostate cancer cells that express wtGp140 were incubated with soluble rExoGp140 and then stained for cellular localization of rExoGp140. Significantly, we localized small levels of rExoGp140 on the cell surface and in the cytoplasm of the cells. The rExoGp140 was detected by staining for the IgG2a Fc domain tag at the C terminal tail of the rExoGp140. These studies suggested that rExoGp140 interacts with an unknown co-receptor, possibly Gp140. We next determine if rExoGp140 interacts with Gp140 and/or signals through Gp140 on the cell surface.

C. Immobilization of recombinant CUB domains on beads and cross-linking to cell-surface receptors. For the functional studies outlined below, rExoGp140 is purified on immobilized Protein A. It can then be multimerized on different surfaces. Alternatively, we can directly immobilize rExoGp140 from the conditioned culture media of the expressing cells onto fluorescent Fluorosphere (4 μM diameter, Molecular Probes) beads using protein A or anti-mouse IgG2a Linker. The latter approach avoids possible denaturation during affinity purification on Protein A and is preferred for functional studies with ECM proteins. Similar approaches can be employed for the immobilization of recombinant Cub1Gp140.

D. Recombinant extracellular CUB1 domains as regulators of Gp140 signaling.

1. Induction of phosphorylation by rExoGp140 and rCubGp140: Studies with rCub1Gp140 and rExoGp140 will determine if they interact with the surface of keratinocytes, PC3 human prostate cancer cell, or 293T cells that have been transfected with wt and mutant forms of Gp140. For these studies, Cub1Gp140 and ExoGp140 are utilized as soluble ligands described above or immobilized on virgin styrene surfaces and/or on Fluorospheres followed by interaction with adherent cells. In one assay, it is determined if surfaces coated with rExoGp140 can induce adhesion of suspended cells or if beads coated with rExoGp140 will bind to the apical surface of adherent cells. In subsequent assays, it is determined if cells that interact with immobilized Gp140 domains induce phosphorylation of Gp140Y734 or SFKY416 at the site of contact of the bead with the adherent cells. These initial studies are followed by larger scale studies for detection of phosphorylation by immunoblotting of Gp140pY734 or SFKpY416.

2. Interactions with rExoGp140 and CUB1 detected by surface plasmon resonance. It is determined if soluble rCub1Gp140 and rExoGp140 interact with each other based on assay with a Biacore 3000 biosensor. ExoGp140 is used as the mobile phase over immobilized exo-Gp140 or Cub1Gp140. Proteins are coupled to CM5 research grade gold biosensor chips using amine coupling chemistry. K_{d}'s are calculated from the best-fit line to a plot of average response at equilibrium verses protein concentration using the BIAevaluation 3.0 software. In general, even low affinity interactions can be measured on the Biacore 3000 as long as the concentration of the soluble mobile ligand is approximately 10× the K_{d} for interaction.

E. Amino acid substitutions in the CUB domains and effects on clustering and signaling. As an alternative to the use of recombinant CUB domains, we will make amino acid substitutions in the CUB domains of human Gp140 or rExoGp140, followed by expression of the mutant cDNA in mouse keratinocytes and analysis of Gp140 cluster assembly, phosphorylation, recruitment of SFK, PKCδ, ZO-1, and Cx43. Site directed mutagenesis of Gp140 will be performed as described for mutations at Gp140Y734F and C689/690S. pBabe-Gp140^{wt} was generated by subcloning the full length human CDCP1 coding sequence from the vector Gp140-pEGFP-N1 (Brown et al., “Adhesion or plasmin regulates tyrosine phosphorylation of a novel membrane glycoprotein p80/gp140/CUB domain-containing protein 1 in epithelia,” J. Biol. Chem. 279: 14772-83, 2004) into the xhol/notl site in pBabe. The Gp140 Y734F mutant (pBabe-Gp140^{Y734F}) was produced by site directed mutagenesis using sequence overlap extension. The following primers were used to introduce an A to T transition at nucleotide 2341 resulting in a Y to F mutation at amino acid residue 734: sense: ctcccatgtgtttgtagtcatcg; anti-sense: cgatgactgcaaacacatgggag. Identity of the mutant was confirmed with direct DNA sequencing. pBabe-Gp140^{wt} and pBabe-Gp140^{Y734F} plasmids were transfected into LA25 cells using jetPEI^{tm} from Polyplus Transfection (San Marcos, Calif.). Stable transfectants were selected with 5 μg/ml puromycin (Sigma).

### Example 5

Characterize the cell signals through Gp140 that regulate assembly of Cx43 in gap junctions. Rationale: Zymosan particles or ActGp140 mAbs interact with the surface of keratinocytes and assemble Gp140 membrane clusters with SFKs, PKC8, and Rac-Tiam1 leading to phosphorylation of Cx43 that regulates GJC. We have utilized both an assembly assay and a disassembly assay to evaluate the signaling path(s) that links Gp140 to Cx43 in GJs. The assembly assay was performed under reduced activity of SFKs, Rac and Ca⁺² concentration so that signals through Gp140 recruited Rac-Tiam 1 into cell-cell contacts that increase Cx43 in functional GJs. In the disassembly assay carried out at elevated SFK, Rac and Ca⁺², signals through Gp140 increase phosphorylation of Cx43 by SFK, PKC and MAPK leading to a decrease in GJC. These results suggest that Gp140 regulates SFKs-dependent phosphorylation of Cx43. Consistently, inhibition of SFK with PP2 increases Gp140 and Cx43 assembly in cell-cell contacts while elevation of SFK with temperature sensitive Src suppresses GJC. Further, expression of Gp140 in LA25 cells is sufficient to reduce both SFK-mediated phosphorylation of Cx43 and reduce the Src-dependent inhibition of GJC. These results suggest that ligation of Gp140 may regulate the activity of SFK and/or the sub-cellular localization of SFKs that control phosphorylation of Cx43.

To place Gp140 into the context of wound repair, Gp140 in cell-cell contacts of following cells or in contact with microbes may retain SFK in cell-cell contacts reducing phosphorylation of Cx43 and increasing GJC. In contrast, loss of Gp140 in leading cells may increase SFK-dependent phosphorylation of Cx43 with loss of GJC. It may also be determined: (a) if Gp140 expression and localization regulates SFK-dependent phosphorylation of Cx43; and (b) if ligation of Gp140 increases SFK-dependent phosphorylation of phospholipase Cγ(PLCγ) to activate PKC-dependent inactivation of RhoGDI, a cytoplasmic inhibitor of Rac (Olofsson, “Rho guanine dissociation inhibitors: pivotal molecules in cellular signaling,” Cell Signal 11: 545-54, 1999).

### Example 6

Determine if knockdown of Gp140 or expression of Gp140 alters phosphorylation of Cx43 in response to zymosan. It is determined if knockdown of Gp140 with siRNA alters phosphorylation of Cx43 by SFKs in response to zymosan. We also screen for possible phosphorylated intermediates in communication between zymosan→Gp140→Cx43.

A. Effects of Gp140 knockdown on phosphorylation of Cx43: The Invitrogen RNAi Designer program was used to generate “Stealth” RNAi that specifically and efficiently knocks down Gp140. The target sequences below are designated with the nucleotide position of the siRNA using the first nucleotide in the AUG codon for the initiating methionine as 1.

Gp140 Stealth 232

| | | |
|---|---|---|
| GCTCTGCCACGAGAAAGCAACATTA | 48% GC | (SEQ ID NO: 1) |

Gp140 Stealth 920

| | | |
|---|---|---|
| CCAGCGTCTCCTTCCTCAACTTCAA | 52% GC | (SEQ ID NO: 2) |

Gp140 Stealth 2338

| | | |
|---|---|---|
| GCAGTCATCGAGGACACCATGGTAT | 52% GC | (SEQ ID NO: 3) |

L6

| | |
|---|---|
| AAGUGUGCACGAUGCAUCGGACAUU | (SEQ ID NO: 4) |

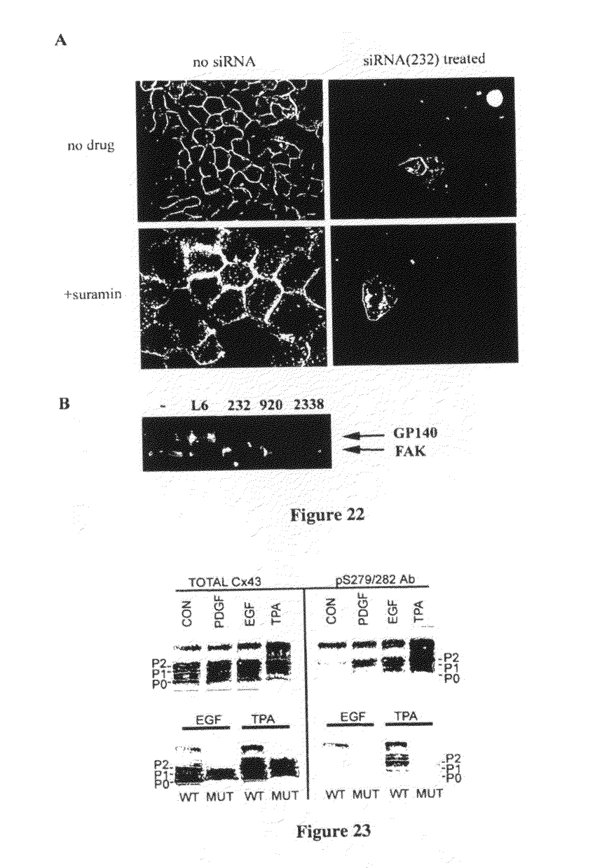

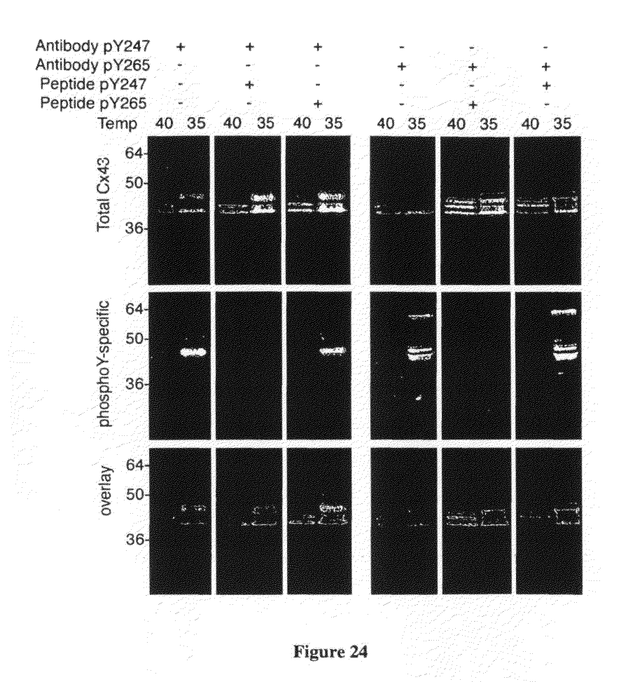

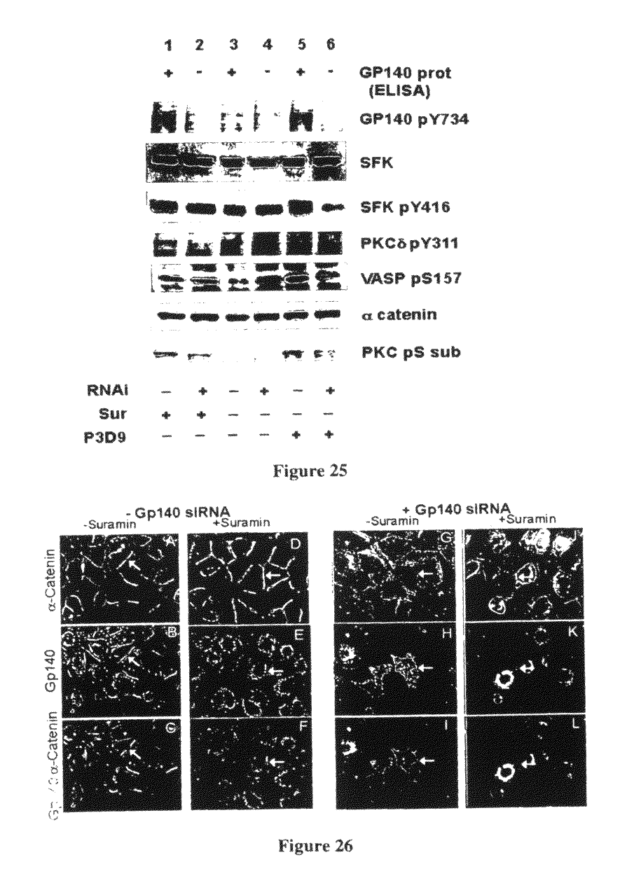

Each Stealth RNAi sequence was nucleoporated (Amaxa) into HKs, then grown for 2 or 4 days, stained and immunoblotted with antibodies against Gp140. As a control for specificity, we also knocked down the L6 tumor antigen utilizing an siRNA we previously prepared. As seen in FIG. 22, Gp140 Stealth RNAi 232, 920 and 2338, but not L6 siRNA, knocked down Gp140 detected by immunofluorescence (A) or immunoblotting (B). The Gp140 siRNA 232 was shown to generate a transient knockdown of Gp140 expression by >90% at both mRNA and protein levels (FIGS. 22A and B). We also prepare a retroviral delivery system for stable knock down of the Gp140/CDCP1 gene using an shRNA with a puromycin selectable marker (OriGene Technologies).

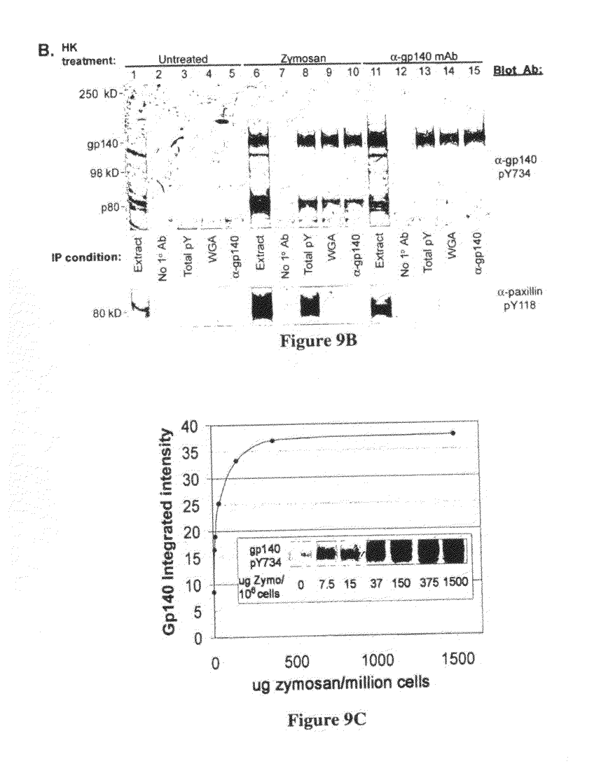

Knockdown (KD) of Gp140 will be performed in a human keratinocyte cell line (HKs) immortalized with HPV E6 and E7. Wild type (wt) HKs and HKGp140KD cells will be incubated with and without zymosan as described in FIG. 9. In wtHKs, this will activate phosphorylation of Gp140Y734, SFKY416, PKCδY311 and paxillinY118. Y-Phosphorylated proteins will be purified by immunoprecipitation with anti-phosphotyrosine mAb (4G10; FIG. 9). We will compare the immunoprecipated pY-proteins by SDS-PAGE and immunoblotting with anti-pY mAb, or antibodies against specific phosphorylated proteins (see paxillin in FIGS. 9 and 10). PP2 inhibition of Y phosphorylation will be used to identify SFK-dependent phosphorylation events. In results discussed in FIG. 2, this approach has already shown that expression of Gp140 in LA25 cells inhibits phosphorylation of Cx43 at Y247 and Y265 by oncogenic Src. We anticipate that knockdown of Gp140 will increase zymosan-induced phosphorylation of Cx43 at Y247 and Y265 resulting in inhibition of GJC. The downregulation of Gp140 in this test system is analogous to the downregulation of Gp140 that occurs in leading cells at the wound edge and may have effects on other SFK-dependent functions including migration via β1 integrins or disassembly of integrin α6β4 in hemidesmosomes.

B. Effects of mutations in Gp140 on phosphorylation of Cx43: We have made Gp140Y734F mutations and found that they inhibit both phosphorylation by Src at this residue and internalization of Gp140 (FIG. 12). As a result of the mutation, Gp140Y734F does not internalize in response to ActGp140 mAb. Hypothetically, failure to internalize Gp140 may either increase or decrease the ability of Gp140 to restrict the sub-cellular localization of SFKs or PKC and effects on phosphorylation of Cx43. We compare wtGp140 and the Gp140Y734F mutant expressed in CWR22 prostate cancer cell that lack endogenous Gp140 mRNA or protein (Carter and Knudsen, unpublished results). We anticipate that Gp140Y734F mutant will alter interactions of SFK and PKCδ at the plasma membrane and interfere with the ability of zymosan or ActGp140 mAb to alter phosphorylation of Cx43. These in vitro results are then be followed by in vivo approaches outlined below.

C. Identification of components that interact with Gp140 in response to zymosan: We identify novel cell components that are phosphorylated in keratinocytes in response to ligation with zymosan. Stimulation with zymosan is expected to increase phosphorylation of Cx43, SFK, and PKCδ as shown in FIG. 9. Adherent keratinocytes are treated without or with zymosan or ActGp140 Mab to induce phosphorylation of proteins and assemble the Triton-resistant Gp140 complex. Alternatively, keratinocytes are adhered to immobilized anti-Gp140 Mab. Extracts of the cells are prepared and immunoprecipitated with antibodies against proteins with phosphorylated tyrosine residues (Mab 4G10). Cell components that are phosphorylated and immunoprecipitated are identified by MS/MS (described below). Immunoprecipitated Gp140 complex is fractionated by SDS-PAGE prior to staining with silver stain and band excision. Protein preparations are identified by LC MS/MS at the FHCRC mass spectrometry facility using protocols we have previously described (Brown et al., “Adhesion or plasmin regulates tyrosine phosphorylation of a novel membrane glycoprotein p80/gp140/CUB domain-containing protein 1 in epithelia,” J. Biol. Chem. 279: 14772-83, 2004; Singh et al., “Identification of connexin-43 interacting proteins,” Cell Commun Adhes 10: 215-20, 2003; Singh et al., “Connexin 43 interacts with zona occludens-1 and -2 proteins in a cell cycle stage-specific manner,” J Biol Chem 280: 30416-21, 2005). After plausible candidates are identified, they are further screened by co-IP and co-immuno labeling.

### Example 7

Characterization of antibodies against phosphorylation sites for Cx43 and Gp140. We have identified 10 specific serine residues in Cx43 that are phosphorylated during gap junctions assembly, in response to kinase activators, or as cells proceed through the cell cycle. Because we have evidence that phosphorylation at S279/282 is involved in cell cycle regulation, we prepared an antibody to phosphorylated S279/282. The specificity of this antibody is shown in FIG. 23. The level of 279/282 phosphospecific antibody binding is increased dramatically upon EGF and TPA treatment and treatment of cells expressing mutant Cx43 with S279 and 282 converted to alanine (MUT) showed essentially no binding even when treated. The antibody is also useful for immunofluorescence (data not shown).

Up to this point, we had mainly utilized tissue culture cells such as NRK and different fibroblast lines. None of these showed significant tyrosine phosphorylation and GM55632 focuses only on serine phosphorylation. Thus, we were surprised when Cx43 from HFKs in culture showed high levels of tyrosine phosphorylation after immunoprecipitation and blot back with nonspecific pY antibodies. We prepared phosphospecific antibodies to pY247 and pY265 and found that, indeed, both show reaction with keratinocytes in culture and in human wounds. The specificity of these antibodies in western immunoblot is shown in FIGS. 23 & 24 and for immunofluorescence in FIGS. 17 & 19.

### Example 8

Characterize the effects of GJC inhibitors, proteasomal inhibitors and antibody effects on wound signaling and migration. We developed an ex vivo wound model utilizing fresh quiescent skin from humans and mice placed in organ culture. Because the model utilizes quiescent epidermis for the starting material, it allows analysis of initial changes in cell signals and adhesion in response to wounding. In contrast, in vitro wound models that utilize cultured keratinocytes start with cells that already have many activated cell signals and proteins. The ex vivo wound model allows the use of pharmacological regulators of signaling proteins in skin wounds followed by assay for biochemical changes by immunoprecipitation and blotting in addition to standard histological studies.

We have evaluated the role of pharmacological and antibody regulators of migration of epidermal keratinocytes in in vitro models of keratinocyte migration (Frank et al., “Laminin 5 deposition regulates keratinocyte polarized and persistent migration,” J. Cell Sci. 117:1351-1363, 2004; Richards et al., “Protein kinase C spatially and temporally regulates gap junctional communication during human wound repair via phosphorylation of connexin43 on serine368,” Journal of Cell Biology 167:555-62, 2004). For example, inhibition of integrin α3β1 or laminin 5, but not integrin α2β1, prevents migration of LKs in migration assays (Frank et al., “Laminin 5 deposition regulates keratinocyte polarized and persistent migration,” J. Cell Sci. 117:1351-1363, 2004). Inhibition of PKCδ and SFK also prevents migration in primary cultures as well as the ex vivo wound outgrowth. Inhibition of GJC with 18α-carbenoxalone (CBX) also prevents wound outgrowth when applied before, but not after wounding (Richards et al., “Protein kinase C spatially and temporally regulates gap junctional communication during human wound repair via phosphorylation of connexin43 on serine368,” Journal of Cell Biology 167:555-62, 2004). This suggests that GJC is required during the initial wound activation to initiate but not maintain the outgrowth. We investigate the role of GJC in initiating the epidermal outgrowth by regulating signaling through Gp140, SFKs and PKC. In particular, we establish the relative order of the activation signals thorough Gp140, MAPK, SFK and PKC in regulating initial wound activation.

The analysis is both by immunohistology of tissue sections and immunoprecipitation and/or immunoblotting of tissue extracts. At time 0, parallel sections (0.5 mm×3-4 mm) of fresh skin explants are cut with a parallel stack of scalpels. The sections are placed in culture of KGM containing 10% serum and cultured for 1-2 minutes to 48 hrs (Frank, unpublished). The sections are fixed (2% formaldehyde, 10 min), stained then mounted between cover slips with a parafilm spacer. The sections are viewed en face as the epithelium migrates from the basement membrane zone (BMZ) over the exposed dermal surface as an epidermal outgrowth or epipole. Alternatively, the sections can be extracted with detergent, followed by immunoprecipitation and immunoblotting as previously described (Brown et al., “Adhesion or plasmin regulates tyrosine phosphorylation of a novel membrane glycoprotein p80/gp140/CUB domain-containing protein 1 in epithelia,” J. Biol. Chem. 279: 14772-83, 2004; Steele et al., “Use of in vitro assays to predict the efficacy of chemopreventive agents in whole animals,” J Cell Biochem Suppl 26: 29-53, 1996; Reddig et al., “Transgenic mice overexpressing protein kinase Cdelta in the epidermis are resistant to skin tumor promotion by 12-O-tetradecanoylphorbol-13-acetate,” Cancer Res 59: 5710-8, 1999).

Inhibition of GJC: We utilize 18α-carbenoxalone (CBX), 18β-glycyrrhetinic acid (βGA), heptanol and octanol as inhibitors of GJC since they are more commonly used in tissue preparations (Largo et al., “Heptanol but not fluoroacetate prevents the propagation of spreading depression in rat hippocampal slices,” J Neurophysiol 77: 9-16, 1997; Rawanduzy et al., “Effective reduction of infarct volume by gap junction blockade in a rodent model of stroke,” J Neurosurg 87: 916-20, 1997; Rodriguez-Sinovas et al., “Enhanced effect of gap junction uncouplers on macroscopic electrical properties of reperfused myocardium,” J Physiol 559: 245-57, 2004) due to their low water solubility. Since CBX and βGA and the alcohols inhibit GJC by different mechanisms (CBX and βGA generally inhibit dye but do not completely block ion permeability while the alcohols limit both) (Rozental et al., “How to close a gap junction channel. Efficacies and potencies of uncoupling agents,” Methods Mol

Biol 154: 447-76, 2001), the results we obtain help us distinguish the regulatory role that GJC plays in wound repair.

We establish the time, location and order of initial activation changes occurring within minutes and hrs at the wound edge. We know that in vivo and ex vivo wounding increases Gp140pY734 and SFKpY416 in LKs between 0 and 6 hrs after injury. We determine when Cx43 is phosphorylated in this window in relation to Gp140 and SFKs in the LKs. Once the descriptive changes in phosphorylation and distribution are mapped in the wound, we determine if inhibition of SFK with PP2 and/or SU6656, inhibition of PKCs with BIM or Go6983, inhibition of MAPK's with PD98059 or activation with TPA impact changes in phosphorylation, subcellular localization, and resistance to extraction with Triton X-100 (4° C.). Suramin increases stable interactions of SFK with Gp140 and phosphorylation of Gp140Y734. TPA activates phosphorylation of Cx43, Gp140, and SFK. These inhibitors-activators allow alterations in interactions of adhesion, Cx43, Gp140, SFKs and PKC in the initial minutes and hrs after wound activation of quiescent skin.

We have already shown that GJC at the time of the wound is necessary for keratinocyte migration (Richards et al., “Protein kinase C spatially and temporally regulates gap junctional communication during human wound repair via phosphorylation of connexin43 on serine368,” Journal of Cell Biology 167:555-62, 2004) to initiate repair, but others have shown that subsequent downregulation can speed healing (Qiu et al., “Targeting connexin43 expression accelerates the rate of wound repair,” Curr. Biol. 13:1697-703, 2003; Kretz et al., “Altered connexin expression and wound healing in the epidermis of connexin-deficient mice,” Journal of Cell Science 116:3443-52, 2003). Therefore, we utilize our keratinocyte migration model and the ex vivo outgrowth wound model to test the effects of PKC, MAPK, SFK and GJC inhibition on keratinocyte migration using the reagents/assays outlined above. Essentially, we extend our previous studies on PKC activation/inhibition (Richards et al., “Protein kinase C spatially and temporally regulates gap junctional communication during human wound repair via phosphorylation of connexin43 on serine368,” Journal of Cell Biology 167:555-62, 2004) to the other kinase systems and place each in a temporal sequence in relation to the regulation of keratinocyte migration. In addition, we utilize our phosphospecific and regular antibodies to determine the localization status of Gp140 and Cx43 in order to correlate it with quantitative changes in the speed and directionality of migration.

For GJC we perform multiple assays to determine whether communication is increased or decreased. The SDS-PAGE band migration pattern of Cx43 is useful. If Cx43 exists as the “P2” form, gap junctions are being assembled (Musil et al., “Biochemical analysis of connexin43 intracellular transport, phosphorylation and assembly into gap junctional plaques,” J. Cell Biol. 115: 1357-1374, 1991). However, with our phosphoantibody tools we can obtain more specific data from immunoblots. Since we know that phosphorylation at S328/330, S365, and S372 is increased in fully assembled junctions and GJC is decreased when S262, S279/282, Y247, Y265, and S368 are phosphorylated, we simply immunoblot and immunostain tissue to obtain information on the functionality of the GJ channels. For example, if S368 is phosphorylated, we have shown that the channels show reduced conductivity (Lampe et al., “Phosphorylation of connexin43 on serine368 by protein kinase C regulates gap junctional communication,” J. Cell Biol. 126:1503-1512, 2000), and others have shown reduced channel open time with tyrosine phosphorylation (Swenson et al., “Tyrosine phosphorylation of the gap junction protein connexin43 is required for pp60^{src}-induced inhibition of communication. Cell Regul 1:989-1002, 1990; Lin et al., “v-Src phosphorylation of connexin 43 on Tyr247 and Tyr265 disrupts gap junctional communication,” J Cell Biol 154:815-27, 2001), so phosphorylation at Y247 and Y265 is informative. In addition, we have also shown that phosphorylation at S328/330 is associated with increased GJ assembly (Cooper et al., “Casein kinase 1 regulates connexin43 gap junction assembly,” J. Biol. Chem. 277: 44962-44968, 2002). Thus, simply knowing the connexin phosphorylation pattern under control and drug treatment conditions allows us to predict the extent of GJC. We readily assay dye permeability of the outgrowth by microinjection of Alexa 488 and 594 (FW 570 and 760) into leading and following cells.

Furthermore, we assay initial gap junction permeability upon wounding using a “cut-loading” procedure where the wound is made in the presence of dye. An advantage of this approach is the ability to observe GJC at “0” time and detect differences in the transfer ability of the LKs and FKs under different drug treatments.

### Example 9

Evaluate the in vivo role of microbe signaling through Gp140 to regulate gap junctions. Our in vitro preliminary studies established that contact of zymosan or S. aureus peptidoglycan with keratinocytes signals through Gp140 to regulate assembly and function of Ws. The assembly of GJ occurs within 30-60 min after contact with zymosan, and we suggest that the assembly contributes to the barrier function of the epidermis that excludes microbes in the wound outgrowth. At a slower rate, signals. through Gp140 also activate SFK- and PKC-dependent phosphorylation of Cx43 that inhibit GJC even 24 hrs after internalization of Gp140. We suggest that these signals lead to disassembly of Gp140 and GJ in leading cells at the wound edge and that facilitates cell migration under and around microbes to cause exclusion.

Significantly, ActGp140 mAbs duplicates most of the signals and functions stimulated by zymosan, suggesting that the functional effects of zymosan on keratinocytes is directly or indirectly through Gp140. We evaluate the function of Gp140 in vivo in responding to microbes (e.g., gram+ bacteria Staphylococcus aureus and/or Streptococcus pyrogenes) in mice and grafts of keratinocytes expressing mutant Gp140 on mice. First, we evaluate the effects of zymosan signaling through Gp140 in wounds of mice. Microbes will be introduced into wounds in skin of mice. We examine the phosphorylation of Gp140 and Cx43 by immunoblotting of epidermis and immunostaining of cryostat sections of mouse skin. Second, we express wild type human Gp140 or mutants of human Gp140 in mouse keratinocytes which are then grown as skin grafts in nude mice (Khavari, “Modelling cancer in human skin tissue,” Nat Rev Cancer 6: 270-80, 2006). The grafts are then treated with zymosan and/or ActGp140 mAb in vivo followed by evaluation of effects on Gp140 and Cx43.

Zymosan effects on phosphorylation of Gp140 and Cx43 in mouse skin. Initial studies are based on immunohistology of cryostat sections of mouse skin and immunoblotting of detergent extracts of epidermis from mice treated with and without zymosan. Mice are treated with zymosan as previously described (Plano et al., “Toxin levels in serum correlate with the development of staphylococcal scalded skin syndrome in a murine model,” Infect Immun 69: 5193-7, 2001). We have previously shown that suramin treatment of mouse epidermis induces phosphorylation and proteolytic cleavage of Gp140 to p80 (Brown et al., “Adhesion or plasmin regulates tyrosine phosphorylation of a novel membrane glycoprotein p80/gp140/CUB domain-containing protein 1 in epithelia,” J. Biol. Chem. 279: 14772-83, 2004). Our initial studies utilize Abs against Gp140pY734, phosphorylated Cx43, NFκB, SFKpY416, and PKCδpY311 that cross react with both mouse and human phosphoproteins. We also prepare rabbit polyclonal antibodies against synthetic peptides from mouse Gp140 for use in immunoprecipitation and immunoblot studies of non-phosphorylated Gp140.

Mouse infection model. The mouse model of infection follows the tape stripping model described by Kugelberg et al. (“Establishment of a superficial skin infection model in mice by using Staphylococcus aureus and Streptococcus pyogenes,” Antimicrob Agents Chemother 49: 3435-41, 2005) The model was developed for superficial skin infections caused by Staphylococcus aureus and Streptococcus pyrogenes, as the most common causative agents of primary skin infections in humans (Chiller et al., “Skin microflora and bacterial infections of the skin,” J Investig Dermatol Symp Proc 6: 170-4, 2001). An alternative skin suture-wound model is also available in which a microbe-impregnated nylon suture is implanted into a scalpel incision through all skin layers. Six to 8 wk old female BALB/c mice are used. Tape stripping the back of the mice 7-10 time causes visibly damaged skin that is red and glistening but without bleeding. A bacterial infection or zymosan inoculation is initiated by placing a 5 μl droplet containing 10⁷ microbes. Mice that are not tape stripped and/or not infected serve as controls.

Studies of human Gp140 function in skin grafts in mice. We evaluate the function of human Gp140 expressed in mouse keratinocytes and grown as skin grafts in nude mice. Using this approach, we express both wild type (wt) and mutant forms of Gp140 in mouse keratinocytes and grow the human keratinocytes as skin grafts on nude mice. First this allows comparisons of wt and mutant Gp140 in the context of normal mouse skin. Second, we compare the effects of zymosan or ActGp140 mAb or NactGp140 mAbs on function of wtGp140 verses Gp140Y734F mutant. This allows us to use our existing library of ActGp140 and NactGp140 mAbs in the mouse system as a way to evaluate their effects in vivo on host defense against microbes. Using this system, we evaluate the effects of Gp140 mutations in response to microbes in mice. Genetically engineered epidermis is regenerated on CB.17 scid/scid mice after gene transfer as described by Cai et al. (Khavari, “Modelling cancer in human skin tissue,” Nat Rev Cancer 6: 270-80, 2006; Cai et al., “Gabl and SHP-2 promote Ras/MAPK regulation of epidermal growth and differentiation,” J Cell Biol 159: 103-12, 2002).

ActGp140 and NActGp140 mAbs as reagents for altering infection by microbes. We utilize the ActGp140 and NActGp140 mouse mAbs in vivo to alter signaling through human Gp140 in skin grafts. In theory, the ActGp140 mAbs that assemble GJs and tight junctions reduce the likelihood of a microbial infection and validate its proposed role in response to microbes. We will utilize the anti-Gp140 mAbs to modulate the microbial response. The mAbs are administered topically at the time of inoculation with microbes. Alternatively, they will be administered via tail vein injection at a dose of 40 mg/kg body.

### Example 10

Signals through Gp140/CDCP1 promote α-catenin in cell-cell contacts. Human keratinocytes were grown in low Ca⁺² (30 μM) in order to disassemble adherens junctions that contain cadherins and α-catenin. The keratinocytes were treated without or with Gp140 siRNA to knockdown expression of Gp140. Without Gp140 siRNA, Gp140 was expressed and overlapped with α-catenin in cell-cell contacts. Activation of Gp140 with suramin (35 μM, 3 hrs) first increased localization of Gp140 with α-catenin in cell-cell contacts (not shown), followed by internalization of Gp140 into the cytoplasm and a dramatic accumulation of α-catenin in cell-cell contacts. However, with Gp140 siRNA, Gp140 was downregulated and α-catenin was not localized in cell-cell contacts either without suramin or with suramin activation. Arrows indicate minor subpopulation of cells where Gp140 was not knocked down surrounded by the majority of Gp140 was knocked down. Only in cell-cell contacts where Gp140 was retained did α-catenin localize in cell-cell contacts. Therefore, we conclude that Gp140 can control the localization of α-catenin in cell-cell contacts in response to outside-in signals from suramin.

### Example 11

Suramin and anti-gp140 mAb (P3D9) signal through Gp140 to activate SFK(s) and PKCS that phosphorylate substrates that may mediate cell-cell adhesion. Keratinocytes were treated without or with Gp140 siRNA (RNAi) to knockdown expression of Gp140. Knockdown of Gp140 protein was confirmed by ELISA assay or immunoblotting with antibodies against Gp140pY734. Activation of Gp140 with either suramin (5 min, 35 mM) or activating anti-Gp140 mAb (P3D9, 5 min) increased phosphorylation of Gp140Y734, SFKY416 and PKCdY311 by SFKs. PKC activity was also increased, as determined by phosphorylation of VaspS157, a substrate for PKGCδ. A protein band that co-migrates with α-catenin was phosphorylated by PKC as determined by immunoblotting with an antibody the binds to protein substrates that are phosphorylated on serine residues by PKC. Knockdown of Gp140 prevented phosphorylation of substrates for SFKs and PKC in response to either suramin or P3D9. We conclude that P3D9 and suramin can signal through Gp140 to control the phosphorylation of cytoplasmic substrates and regulate the assembly of α-catenin in cell-cell contacts.

The foregoing is illustrative of the present invention, and is not to be construed as limiting thereof. The invention is defined by the following claims, with equivalents of the claims to be included therein.

1. A method of modulating cell junctions of cells comprising administering to said cells a Gp140 agonist or antagonist in an amount effective to modulate cell junctions in said cells.

2.-10. (canceled)

11. The method of claim 1, wherein said cells are exposed to stress.

12. The method of claim 11, wherein said administering step is carried out by administering a Gp140 agonist, and said agonist is an activating Gp140 antibody zymosan Staphylococcus aureus peptidoglycan or suramin.

13.-15. (canceled)

16. The method of claim 11, wherein said administering step is carried out by administering a Gp140 antagonist, and said antagonist is a non-activating Gp140 antibody or a non-coding RNA.

17. (canceled)

18. The method of claim 11, wherein said stress is selected from the group consisting of: a cardiac dysfunction and a brain injury.

19. The method of claim 18, wherein said stress is a brain injury, and said brain injury is a stroke.

20. A method of treating a cell junction disorder or a cancer in a subject in need thereof, comprising administering to said subject a Gp140 agonist or antagonist in an amount effective to treat said cell junction disorder or said cancer.

21. The method of claim 20, wherein said subject is in need of treatment for a cell junction disorder selected from the group consisting of: a connexin disorder, a tight junction disorder, an adherens junction disorder, a focal adhesion disorder and a desmosome disorder.

22. The method of claim 20, wherein said subject is in need of treatment for a cell junction disorder that is a connexin disorder selected from the group consisting of: skin disorders, nervous system disorders, heart disorders and muscle disorders.

23.-30. (canceled)

31. The method of claim 20, wherein said subject is in need of treatment for cancer and said agonist or antagonist is administered in an amount effective to increase cell junction number and/or function in the cells of said cancer.

32. The method of claim 20, wherein said subject is in need of treatment for cancer selected from the group consisting of: skin cancer, lung cancer, prostate cancer, breast cancer and colon cancer.

33. The method of claim 20, wherein said subject is in need of treatment for cancer and said cancer is metastatic melanoma.

34. A method of treating a wound or an inflammatory disease in a subject in need thereof, comprising administering to said subject a Gp140 agonist or antagonist in an amount effective to treat said wound or said inflammatory disease.

35. The method of claim 34, wherein said administering step is carried out by administering a Gp140 agonist, and said agonist is an antibody that specifically binds to an extracellular domain of Gp140 and activates Gp140, zymosan, Staphylococcus aureus peptidoglycan, or suramin.

36.-38. (canceled)

39. The method of claim 34, wherein said administering step is carried out by administering a Gp140 antagonist, and said antagonist is an antibody that specifically binds to an extracellular domain of Gp140 and does not activate Gp140, or a non-coding RNA.

40.-47. (canceled)

48. A composition comprising a Gp140 agonist or a GP140 antagonist and a pharmaceutically acceptable carrier.

49. The composition of claim 48, wherein said Gp140 agonist is selected from the group consisting of: an activating antibody of Gp140, zymosan, Staphylococcus aureus peptidoglycan, and suramin.

50.-51. (canceled)

52. The composition of claim 48, wherein said composition is a cream or ointment.

53.-54. (canceled)

55. The composition of claim 48, wherein said antagonist is a non-activating or blocking antibody of Gp140.

56.-59. (canceled)


SEQUENCE LISTING (4 sequences)

SEQ ID NO: 1 - DNA, 25 bp, Artificial
  Gp140 Stealth 232 RNAi sequence
gctctgccacgagaaagcaacatta

SEQ ID NO: 2 - DNA, 25 bp, Artificial
  Gp140 Stealth 920 RNAi sequence
ccagcgtctccttcctcaacttcaa

SEQ ID NO: 3 - DNA, 25 bp, Artificial
  Gp140 Stealth 2338 RNAi sequence
gcagtcatcgaggacaccatggtat

SEQ ID NO: 4 - DNA, 25 bp, Artificial
  L6 siRNA sequence
aagugugcacgaugcaucggacauu